(19) United States
(12) Patent Application Publication
(10) Pub. No.: US 2025/0313887 A1
(43) Pub. Date: Oct. 9, 2025

(54) SUPPRESSION OF TARGETED APTAMER CLUSTER

(71) Applicant: Eisai R&D Management Co., Ltd., Tokyo (JP)

(72) Inventor: Yogo Sakakibara, Kobe-Shi, Hyogo (JP)

(73) Assignee: Eisai R&D Management Co., Ltd., Tokyo (JP)

(21) Appl. No.: 18/872,792

(22) PCT Filed: Jul. 3, 2023
(86) PCT No.: PCT/JP2023/024609

§ 371 Date: Dec. 6, 2024

Related U.S. Application Data
(60) Provisional application No. 63/358,365, filed on Jul. 5, 2022.

(51) Int. Cl. C12Q 1/6869; C12N 15/115; C12Q 1/6876

(52) U.S. Cl. CPC C12Q 1/6869; C12N 15/115; C12Q 1/6876; C12N 2310/16; C12N 2330/31

(57) ABSTRACT

The present disclosure, at least in part, relates to methods of sequencing low abundance aptamers from an aptamer library, the method comprising: (i) amplifying a plurality of aptamers capable of binding to one or more target molecules in a sample by emulsion PCR to generate an aptamer library; (ii) sequencing the aptamer library; and (iii) contacting a plurality of antisense oligonucleotides (ASOs) targeting high abundance aptamers of the aptamer library with the aptamer library to form a mixture, wherein contacting the ASOs with the aptamer library results in inactivation of the high abundance aptamers of the aptamer library.

### TECHNICAL FIELD

The present invention generally relates to a method of sequencing low abundance aptamers from an aptamer library.

### BACKGROUND ART

Enriched aptamers are subject to sequencing analysis to obtain sequencing information. However, large amount of sequence information was lost for aptamers of low abundance in the enriched aptamer library.

### CITATION LIST

### Patent Literature

[PTL 1] US 2006/0160763

### Non Patent Literature

[NPL 1] Keefe et al., Aptamers as therapeutics. Nat. Rev. Drug Discov. 2010;9:537-550.
[NPL 2] Jayasena S. D., Aptamers: An emerging class of molecules that rival antibodies in diagnostics. Clin. Chem. 1999;45:1628-1650.
[NPL 3] Tuerk et al., Systematic evolution of ligands by exponential enrichment: RNA ligands to bacteriophage T4 DNA polymerase. Science. 1990;249:505-510.
[NPL 4] Ellington et al., In vitro selection of RNA molecules that bind specific ligands. Nature. 1990;346:818-822.
[NPL 5] SELEX-A (Registered Trademark) evolutionary method to generate high-affinity nucleic acid ligands. Biomolecular Engineering, 24 (4):381-403; Klug & Famulok (1994).
[NPL 6] All you wanted to know about SELEX. Molecular Biology Reports, 20:97-107; Darmostuk et al. (2015).
[NPL 7] Current approaches in SELEX: An update to aptamer selection technology. Biotechnology Advances, 33 (6):1141-1161.
[NPL 8] Schutze et al., Probing the SELEX Process with Next-Generation Sequencing, PLOS One. 2011; 6 (12):e29604.
[NPL 9] Hao et al., Introduction of Aptamer, SELEX, and Different SELEX Variants, Aptamers for Medical Applications pp 1-30.
[NPL 10] Chai et al., Principle of Emulsion PCR and Its Applications in Biotechnology, J Anim Reprod Biotechnol 2019;34:259-266.
[NPL 11] Shao et al., Emulsion PCR: A High Efficient Way of PCR Amplification of Random DNA Libraries in Aptamer Selection, PLOS ONE 6(9): e24910.
[NPL 12] Duffy et al., Modified nucleic acids: replication, evolution, and next-generation therapeutics, BMC Biology volume 18, Article number: 112 (2020).
[NPL 13] Hollenstein (2015) Generation of long, fully modified, and serum-resistant oligonucleotides by rolling circle amplification. Organic Biomolecular Chemistry, 13:9829.
[NPL 14] Kong et al. (2016). Generation of Synthetic Copolymer Libraries by Combinatorial Assembly on Nucleic Acid Templates. ACS Combinatorial Science, 18:355-370.
[NPL 15] Abraham et al., A quick and effective in-house method of DNA purification from agarose gel, suitable for sequencing, 3 Biotech. 2017 July; 7(3): 180.
[NPL 16] Leonard et al., Basic Methods in Molecular Biology, 1986, SECTION 5-5-Agarose Gel Electrophoresis, pages 58-61.

### SUMMARY OF INVENTION

### Technical Problem

The object of the present invention is to provide methods of sequencing low abundance aptamers from an aptamer library.

### Solution to Problem

The present disclosure, at least in part, relates to methods for mining the sequence information of low abundance aptamers from an aptamer library. An aptamer library comprises high abundance aptamers (e.g., aptamers with a sequence frequency level higher than a percentage in the sequencing reaction) and low abundance aptamers (e.g., aptamers with a sequence frequency level lower than a percentage in the sequencing reaction). In some embodiments, during sequencing reaction, the high abundance aptamers conceal the sequence information of low abundance aptamers from being effectively analyzed due to low reads of the low abundance aptamers (e.g., low reads that fall below the detection limit). In some embodiments, the present disclosure provides methods for depleting the high abundance aptamers (e.g., by antisense oligonucleotides (ASOs) targeting the high abundance aptamers) such that the low abundance aptamers can be effectively amplified (e.g., by emulsion PCR) and sequenced. In some embodiments, the methods can be described as suppression of targeted aptamer cluster (STAC).

In some aspects, the present disclosure provides a method of sequencing low abundance aptamers from an aptamer library, the method comprising: (i) amplifying a plurality of aptamers capable of binding to one or more target molecules in a sample by emulsion PCR to generate an aptamer library; (ii) sequencing the aptamer library; and (iii) contacting a plurality of antisense oligonucleotides (ASOs) targeting high abundance aptamers of the aptamer library with the aptamer library to form a mixture; wherein contacting the ASOs with the aptamer library results in inactivation of the high abundance aptamers of the aptamer library.

In some embodiments, the method further comprises selecting the plurality of aptamers capable of binding to one or more target molecules in a sample in steps (a)-(c): (a) contacting a plurality of candidate aptamers with a sample comprising one or more target molecules to form a composition comprising a plurality of aptamer-target molecule complexes; (b) purifying the plurality of aptamer-target molecule complexes; and (c) extracting the plurality of aptamers capable of binding to one or more target molecules from the aptamer-target molecule complexes.

In some embodiments, the method further comprises repeating the steps (a)-(c) and (i)-(iii), and wherein the mixture obtained from step (iii) comprises the plurality of candidate aptamers when repeating step (a). In some embodiments, the method is repeated at least three times. In some embodiments, the method further comprises sequencing the aptamer library obtained from step (iii).

In some embodiments, the ASOs comprise modified nucleotides.

In some embodiments, sequencing low abundance aptamers from an aptamer library comprises next generation sequencing (NGS).

In some embodiments, the sample is a biological sample. In some embodiments, the biological sample is serum, plasma, cerebral-spinal fluid (CSF), urine, amniotic fluid, bone marrow, bronchoalveolar lavage fluid, buccal swab, feces, gastrointestinal fluid, liposuction sample, saliva, milk, nasal swab, peritoneal fluid, semen, sputum, synovial fluid, tears, vaginal fluid, tissue biopsy, cell lysates, vaginal fluid, tissue biopsy, or cell lysates, cultured. In some embodiments, the biological sample comprises target molecules including nucleic acids, proteins, polypeptides, carbohydrates, lipids, or a combination thereof. In some embodiments, the biological sample is not denatured.

In some embodiments, the high abundance aptamers in the aptamer library are aptamers having a sequence frequency level of higher than 0.05% in the sequencing reaction in step (ii). In some embodiments, the high abundance aptamers in the aptamer library are aptamers having a sequence frequency level of higher than 0.1% in the sequencing reaction in step (ii). In some embodiments, the high abundance aptamers in the aptamer library are aptamers having a sequence frequency level of higher than 0.15% in the sequencing reaction in step (ii). In some embodiments, the high abundance aptamers in the aptamer library are aptamers having a sequence frequency level of higher than 0.2% in the sequencing reaction in step (ii). In some embodiments, the high abundance aptamers in the aptamer library are aptamers having a sequence frequency level of higher than 0.5% in the sequencing reaction in step (ii).

In some embodiments, step (b) comprises subjecting the composition to electrophoresis in a first electrophoresis medium in a first direction to obtain a portion of the first electrophoresis medium that comprises the aptamer-target molecule complexes.

In some embodiments, step (b) further comprises subjecting the portion of the first electrophoresis medium to electrophoresis in a second electrophoresis medium in a second direction to obtain a portion of the second electrophoresis medium that comprises the aptamer-target molecule complexes.

In some embodiments, the first electrophoresis medium is a first agarose gel. In some embodiments, the second electrophoresis medium is a second agarose gel.

In some embodiments, the first and the second electrophoresis media comprise sodium ion, potassium ion, lithium ion, ammonium ion or any combination thereof at a concentration of between 100 mM and 200 mM. In some embodiments, the sodium ion is in the form of sodium chloride.

In some embodiments, the first and the second electrophoresis media comprise magnesium ion, calcium ion, copper ion, zinc ion or any combination thereof at a concentration of 10 mM or less. In some embodiments, the first and the second electrophoresis media comprise magnesium ion, calcium ion, copper ion, zinc ion or any combination thereof at a concentration of between 0.5 mM and 2 mM. In some embodiments, the first and the second electrophoresis media comprise magnesium ion, calcium ion, copper ion, zinc ion or any combination thereof at a concentration of 1 mM. In some embodiments, the magnesium ion is in the form of magnesium chloride.

In some embodiments, step (iii) further comprises excising the portion of the first electrophoresis medium comprising the aptamer-target molecule complexes from the rest of the first electrophoresis medium. In some embodiments, the portion of the first electrophoresis medium was fitted to a well in the second electrophoresis medium for performing the electrophoresis in the second direction.

In some embodiments, the electrophoresis in the first direction and the second direction are performed in a temperature between 10° C. and 20° C.

In some embodiments, the plurality of candidate aptamers are single stranded DNAs (ssDNA), double stranded DNAs (dsDNA), single stranded RNAs, or peptides. In some embodiments, the plurality of candidate aptamers are single stranded DNAs (ssDNA).

In some embodiments, each of the plurality of the candidate aptamers comprises modified nucleotide. In some embodiments, each of the plurality of the candidate aptamers comprise one or more 5-tryptamino-uracil in place of thymine.

In some embodiments, each of the plurality of the candidate aptamers are labeled. In some embodiments, each of the plurality of the candidate aptamers is fluorescent-labeled.

In some embodiments, the method further comprises excising the portion of the second electrophoresis medium containing the aptamer-target molecule complexes from the rest of the second electrophoresis medium and extracting the aptamer-target molecule complexes from the portion of the second electrophoresis medium prior to step (c).

In some embodiments, the method further comprises denaturing and renaturing the aptamer library before contacting the ASOs with the aptamer library.

In some embodiments, the method further comprises denaturing and renaturing the aptamer library after contacting the ASOs with the aptamer library.

### Advantageous Effects of Invention

The present invention provides a method of sequencing low abundance aptamers from an aptamer library. The method enables data mining and deeper analysis of an aptamer library.

### BRIEF DESCRIPTION OF DRAWINGS

FIG. 1 shows, high abundance aptamer clusters conceal the sequence in-formation of low abundance aptamers from efficient NGS analysis and sequence mining of an aptamer library. The exemplary plot on the top shows typical NGS data of an aptamer library demonstrating that low abundance aptamers are below detection limit and difficult to analyze due to low reads. The exemplary plot on the bottom shows NGS data of an aptamer library wherein high abundance aptamers have been depleted, which resulted in increased reads of low abundance aptamers.

FIG. 2 shows an exemplary strategy for sequence depletion in an aptamer library. Antisense oligo (ASO) targeting to high abundance aptamers induces structural changes of the targeted aptamers resulting in deactivation of these aptamers, thereby inhibiting interaction of the high abundance aptamers with their respective target molecules.

FIGS. 3 and 4 are graphs showing ASOs depletion of high abundance aptamers in an aptamer library resulted in increase in percentage reads of the low abundance aptamers. FIG. 3 shows exemplary results of efficient and specific ASO deactivation of a high abundance aptamers. The top 18 (1-9, 11-14, and 16-20) high abundance aptamers were specifically and efficiently depleted by ASO modeling and remaining sequences showed increase in the percentage of reads. In order to test knock down specificity, no ASOs were designed to target aptamers #10 and #15. Therefore, after contacting the aptamer library with the ASOs, aptamers #10 and #15 showed increase in percentage reads.

FIGS. 3 and 4 are graphs showing ASOs depletion of high abundance aptamers in an aptamer library resulted in increase in percentage reads of the low abundance aptamers. FIG. 4 shows knocking down of high abundance aptamers resulted in creating more space for NGS analysis for low abundance sequences.

FIGS. 5-7 show exemplary fold changes in sequence frequency after different PCR procedures. The top 100 aptamers are shown. FIGS. 5-6 show exemplary data that compared the PCR product between emulsion PCR (FIG. 5) and general PCR (FIG. 6) methods obtained by duplicating the same aptamer library at the same time.

FIGS. 5-7 show exemplary fold changes in sequence frequency after different PCR procedures. The top 100 aptamers are shown. FIGS. 5-6 show exemplary data that compared the PCR product between emulsion PCR (FIG. 5) and general PCR (FIG. 6) methods obtained by duplicating the same aptamer library at the same time.

FIGS. 5-7 show exemplary fold changes in sequence frequency after different PCR procedures. FIG. 7 shows data for PCR product check by PAGE. Emulsion conditions are as follows: 1; combination of sonication and voltex, 2; voltex, 3; string by magnetic stirrer bar, 4; general PCR.

FIGS. 8 and 9 show exemplary NGS analysis of aptamer libraries. FIG. 8 shows exemplary NGS analysis of aptamer libraries with or without depletion of high abundance aptamers using 18 different ASOs. Sequences 10 and 15 were not targeted by any ASO in this experiment to investigate ASO specificity.

FIGS. 8 and 9 show exemplary NGS analysis of aptamer libraries. FIG. 9 shows exemplary NGS analysis of aptamer libraries with or without depletion of high abundance aptamers using 52 different ASOs. Non-targeted sequences are shown after sequence 53 for comparison.

FIGS. 10 and 11 show change in sequence frequency after aptamer depletion by the addition of 52 different ASOs is shown for top 500 sequences. FIG. 10 shows that ASOs were added before library denaturation to strongly remove target sequences. Data is drawn in terms of aptamer sequences to show change in sequence frequency with or without ASOs. Sequences that were not targeted by ASOs but decreased due to the addition of ASOs are shown separately as non-specific effects.

FIGS. 10 and 11 show change in sequence frequency after aptamer depletion by the addition of 52 different ASOs is shown for top 500 sequences. FIG. 11 shows that ASOs were added after library denaturation for more specific removal of target sequences. Sequences that were not targeted by ASOs but decreased due to the addition of ASOs are shown separately as non-specific effects.

FIGS. 12 shows exemplary NGS data indicating the number of sequences that were detected as a result of ASO-dependent depletion of high abundance aptamers.

### DESCRIPTION OF EMBODIMENTS

The present disclosure, at least in part, relates to methods for mining the sequence information of low abundance aptamers from an aptamer library. An aptamer library comprises high abundance aptamers (e.g., aptamers with a sequence frequency level higher than a percentage in the sequencing reaction) and low abundance aptamers (e.g., aptamers with a sequence frequency level lower than a percentage in the sequencing reaction). In some embodiments, during sequencing reaction, the high abundance aptamers conceal the sequence information of low abundance aptamers from being effectively analyzed due to low reads of the low abundance aptamers (e.g., low reads that fall below the detection limit). In some embodiments, the present disclosure provides methods for depleting the high abundance aptamers (e.g., by antisense oligonucleotides (ASOs) targeting the high abundance aptamers) such that the low abundance aptamers can be effectively amplified (e.g., by emulsion PCR) and sequenced.

The term “aptamer” as used herein, refers to oligonucleotide (e.g., single-stranded DNA (ssDNA) or single-stranded RNA (ssRNA) molecule that can specifically bind to a target molecule. In some embodiments, an aptamer is a single-stranded DNA aptamer. In some embodiments, an aptamer comprises between 20 and 60 nucleotides, between 25 and 55 nucleotides, between 30 and 50 nucleotides, between 35 and 45 nucleotides, between 20 and 50 nucleotides, between 20 and 40 nucleotides, between 25 and 40 nucleotides, between 20 and 30 nucleotides, between 30 and 40 nucleotides, between 30 and 60 nucleotides, between 40 and 60 nucleotides, or between 50 and 60 nucleotides. In some embodiments, target molecules of an aptamer include proteins, peptides, carbohydrates, small molecules, toxins, and cells (e.g., live cells). An aptamer binds to its target with high affinity, selectivity and specificity (see., e.g., Non Patent Literature 1 and 2). Rather than primary sequence, aptamer binding is determined by its tertiary structure. Target recognition and binding of an aptamer involve three-dimensional, shape-dependent interactions as well as hydrophobic interactions, base-stacking, and intercalation. Aptamers offer advantages over antibodies as they can be engineered completely in vitro, are readily produced by chemical synthesis, possess desirable storage properties, and elicit little or no immunogenicity in therapeutic applications.

The concept of in vitro evolution of aptamers was introduced in 1990 and termed Systematic Evolution of Ligands by EXponential enrichment, or SELEX (Non Patent Literature 3 to 7). SELEX combines the rules of combinatorial library screening with in vitro evolution for enriching aptamers (e.g., DNA aptamers) against a wide range of target molecules. The SELEX process involves three interconnected steps: (i) the repeated incubation of a plurality of candidate aptamers with a target molecule to allow binding of high-affinity aptamers; (ii) the separation of high-from low-affinity binders and/or non-binders; and (iii) amplification of high-affinity binders utilizing polymerase chain reaction (PCR). This process is repeated until the high-affinity aptamers are enriched in the selection pool.

In some embodiments, after each round of selection and amplification, the resulting aptamers can be further analyzed, e.g., by binding assay, diversity assay, or sequencing. In conventional SELEX, the terminal aptamer library after the selection is cloned and 30-100 representatives are sequenced with Sanger sequencing. In that respect, the correct identification of candidate aptamers is a key point for the overall selection success. The aptamer isolation process retrieves a nucleic acid library enriched with sequences binding specifically with a target. In the classic method, the resulting enriched nucleic acid pool is cloned, and 30-100 clones are than Sanger-sequenced with the aim of determining a few aptamer candidates for further detailed characterization. Cluster analysis assists in the identification of aptamer candidates. Usually, the most abundant species or representatives of the largest clusters among the sequenced pool are believed to be potential aptamers. Many aptamers have been isolated and characterized using this understandable approach. In the last decade, next-generation sequencing (NGS) technologies have progressed to become a popular method for high-throughput aptamer sequencing. In some embodiments, the resulting aptamers from each selection round are sequenced by next generation sequencing (NGS). However, in some cases, the most abundant aptamer sequences identified by NGS do not show the best binding to the target, probably due to PCR bias (Non Patent Literature 8). Further, in some embodiments, the sequence information for aptamers at low level may be lost due to low reads below detection level.

### I. Suppression of Targeted Aptamer Cluster

The present disclosure, at least in part, provides methods for suppression of targeted aptamer clusters (e.g., high abundance aptamers with a sequence frequency level higher than a percentage in the sequencing reaction) such that the sequence frequency of the low abundance aptamer increases and can be detected. In some embodiments, the methods provided herein enables data mining and deeper analysis of an aptamer library.

In some aspects, the present disclosure provides a method of sequencing low abundance aptamers from an aptamer library, the method comprising: (i) amplifying a plurality of aptamers capable of binding to one or more target molecules in a sample by emulsion PCR to generate an aptamer library; (ii) sequencing the aptamer library; and (iii) contacting a plurality of antisense oligonucleotides (ASOs) targeting high abundance aptamers of the aptamer library with the aptamer library to form a mixture; wherein contacting the ASOs with the aptamer library results in inactivation of the high abundance aptamers of the aptamer library.

In some embodiments, a plurality of aptamers capable of binding to one or more target molecules in a sample can be a plurality of aptamers enriched for a target molecule by any suitable known method, e.g., by conventional SELEX or any variation thereof, e.g., the SELEX methods as described by Non Patent literature 9.

In some embodiments, the plurality of aptamers capable of binding to one or more target molecules in a sample are amplified to form an aptamer library prior to being subjected to sequencing. In some embodiments, the plurality of aptamers comprises aptamers at different abundance (i.e., sequence order information). In some embodiments, aptamers bind to target molecule with higher affinity are present in the plurality of aptamers capable of binding to the target molecule at a higher abundance than aptamers that bind to the target molecule with lower affinity. In some embodiments, it is crucial to preserve the sequence order information during amplification such that the sequencing step can correctly identify the high abundance aptamers in the aptamer library. In some embodiments, any amplification method can be employed by the methods described herein provided that the amplification method (e.g., PCR) is capable of preserving the sequence order information in the plurality of aptamers capable of binding to one or more target molecules (e.g., biomolecules) after forming the aptamer library by amplification.

In some embodiments, the present disclosure, at least in part, is based on the discovery that amplification of the plurality of aptamers capable of binding to one or more target molecules in a sample by emulsion PCR preserves the correct sequence order information. The term “emulsion PCR”, as used herein, refers to PCR reaction performed on aqueous droplets emulsified in oil phase of water in oil emulsion. In some embodiments, the aqueous droplets serve as miniaturized “reactors” for each PCR reaction, and are physically separated from each other without exchange of macromolecules, especially the PCR products. Individual DNA molecules are compartmentalized into these distinct reaction droplets, allowing their amplification independent of one another. With emulsion PCR, the formation of unproductive chimeras and other by-products are avoided, and the overall amplification bias is reduced. (See, e.g., Non Patent Literature 10 and 11). In some embodiments, the emulsion PCR reaction is prepared by mixing the PCR solution with emulsion oil. In some embodiments, the emulsion oil comprises oil (e.g., mineral oil). In some embodiments, the emulsion oil comprises oil (e.g., mineral oil) at a concentration of between 90% and 98%, between 91% and 97%, between 92% and 96%, between 93% and 95%, between 94% and 96%, between 90% and 95%, between 91% and 96%, between 92% and 96%, between 93% and 96%, between 94% and 97%, or between 95% and 96%. In some embodiments, the emulsion oil comprises oil (e.g., mineral oil) at 95.05%. In some embodiments, the emulsion PCR reaction is vigorously shaken to form the water-in-oil emulsion droplet. In some embodiments, prior to performing the amplification step, a pre-run of the emulsion PCR is performed at fixed cycles, and the PCR products are analyzed to select a proper PCR cycle that provides a single population of PCR product (e.g., shown as a clear single band on DNA electrophoresis). In some embodiments, the emulsion PCR is performed at 5, 6, 7, 8, 9, 10,11, 12, 13, 14, 15, 16, 17, 18, 19, 20, 21, 22, 23, 24, 25, or more cycles for amplifying the plurality of aptamers.

In some embodiments, the method further comprising sequencing the aptamers in the aptamer library generated from the emulsion PCR amplification. The term “sequencing”, as used herein, with respect to a nucleic acid, refers a process of determining the nucleotide order of a given nucleic acid fragment. Methods of sequencing nucleic acids (e.g., aptamers) are known in the art, which include but are not limited to basic sequencing (e.g., Maxam-Gilbert sequencing), chain-termination sequencing (e.g., Sanger sequencing), large-scale sequencing and de novo sequencing (e.g., shotgun sequencing), next generation sequencing (e.g., Single-molecule real-time sequencing, Ion Torrent sequencing, Pyrosequencing, Sequencing by synthesis (e.g., MiSeq), Combinatorial probe anchor synthesis, Sequencing by ligation (SOLID sequencing), Nanopore Sequencing, GenapSys Sequencing, or Chain termination (Sanger sequencing), long-read sequencing, Short-read sequencing methods (e.g., Massively parallel signature sequencing (MPSS), Polony sequencing, 454 pyrose-quencing, Illumina (Solexa) sequencing, DNA nanoball sequencing, Heliscope single molecule sequencing, Microfluidic Systems). In some embodiments, the aptamers are sequenced by next generation sequencing (e.g., MiSeq).

In some embodiments, the sequencing data (e.g., NGS data) of the aptamer library comprises a high frequent region corresponding to high abundance aptamers in the aptamer library, and a low frequent region corresponding to low abundance aptamers in the aptamer library. In some embodiments, high abundance aptamers are aptamers having a sequence frequency level of higher than 0.05%, higher than 0.06%, higher than 0.07%, higher than 0.08%, higher than 0.09%, higher than 0.10%, higher than 0.11%, higher than 0.12%, higher than 0.13%, higher than 0.14%, higher than 0.15%, higher than 0.16%, higher than 0.17%, higher than 0.18%, higher than 0.19%, higher than 0.20%, higher than 0.21%, higher than 0.22%, higher than 0.23%, higher than 0.24%, higher than 0.25%, higher than 0.26%, higher than 0.27%, higher than 0.28%, higher than 0.29%, higher than 0.30%, higher than 0.31%, higher than 0.32%, higher than 0.33%, higher than 0.34%, higher than 0.35%, higher than 0.36%, higher than 0.37%, higher than 0.38%, higher than 0.39, higher than 0.40%, higher than 0.41%, higher than 0.42%, higher than 0.43%, higher than 0.44%, higher than 0.45%, higher than 0.46%, higher than 0.47%, higher than 0.48%, higher than 0.49, higher than 0.50%, or higher. In some embodiments, sequence information for low abundance aptamers is below detection limit such that the sequence information of these aptamers are unattainable. In some embodiments, the high abundance aptamer conceals low abundance aptamers from efficient sequencing analysis.

The present disclosure, at least in part, is based on the discovery that by removing the high abundance aptamers from the aptamer library, the sequence information of the low abundance aptamers can be obtained in the next round of selection. In some aspects, the method provided herein further comprises contacting a plurality of contacting a plurality of antisense oligonucleotides (ASOs) targeting high abundance aptamers of the aptamer library with the aptamer library to form a mixture. In some embodiments, the ASOs targeting high abundance aptamers of the aptamer library are designed according to the sequence information of the high abundance aptamers obtained from the sequencing reaction.

As used herein, the term “antisense oligonucleotide (ASO)” refers to an oligomeric compound, at least a portion of which is at least partially complementary to an aptamer to which it hybridizes, wherein such hybridization results in at least one antisense activity (e.g., inactivation of the aptamer).

In some embodiments, an ASO targeting an aptamer are designed to cause conformation change of the aptamer such that it can no longer binds to its target molecule. In some embodiments, an ASO targeting an aptamer comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides to an aptamer. In some embodiments, an ASO targeting aptamer comprises a region of complementarity to any one of the high abundance aptamers in the aptamer library. In some embodiments, an ASO targeting an aptamer comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides to the nucleotide sequence of any one of the high abundance aptamers in the aptamer library. In some embodiments, an ASO targeting an aptamer comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides, except for at least 1, at least 2, at least 3, at least 4, or at least 5 mismatches of the aptamer.

ASOs may be of a variety of different lengths. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, an ASO is 25-30 nucleotides in length, 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, or 21 to 23 nucleotides in length. In some embodiments, an ASO for purposes of the present disclosure specifically hybridizes (e.g. has complementarity to) to an aptamer when binding of the ASO to the aptamer mRNA interferes with the normal function of the aptamer to cause a loss of activity (e.g., inhibiting binding to target molecule of the aptamer), and there is a sufficient degree of complementarity to avoid non-specific binding of the antisense oligonucleotide sequence to non-target aptamers under conditions in which avoidance of non-specific binding is desired, e.g., under conditions in which the assays are performed under suitable conditions of stringency. Thus, in some embodiments, an ASO may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of an aptamer. In some embodiments, an ASO need not be 100% complementary to that of the consecutive region of the aptamer to be specifically hybridizable or specific for the aptamer.

In some embodiments, one or more of the thymine bases (T's) in any one of the ASO may optionally be uracil bases (U's), and/or one or more of the U's may optionally be T's.

An antisense oligonucleotide described herein may be modified, e.g., comprise a modified sugar moiety, a modified internucleotide linkage, a modified nucleobase, a modified nucleotide, and/or (e.g., and) combinations thereof.

In some embodiments, an antisense oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, an antisense oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified nucleoside. In some embodiments, an antisense oligonucleotide comprises one or more 2′-O-methoxyethyl (2′-MOE) modified nucleoside. In some embodiments, each of the nucleosides of the antisense oligonucleotide is a 2′-O-methoxyethyl (2′-MOE) modified nucleoside. In some embodiments, an antisense oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO 2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. No. 7,101,993; 7,399,845 and 7,569,686, each of which is herein incorporated by reference in its entirety.

In some embodiments, an antisense oligonucleotide comprises a modified nucleoside disclosed in one of the following US Patents or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17,2015, and entitled “Oligonucleotide Analogues And Methods Utilizing The Same”, the entire contents of each of which are incorporated herein by reference.

In some embodiments, an antisense oligonucleotide may contain a phosphorothioate or other modified internucleotide linkage. In some embodiments, an antisense oligonucleotide comprises phosphorothioate internucleotide linkages. In some embodiments, an antisense oligonucleotide comprises phosphorothioate internucleotide linkages between at least two nucleotides. In some embodiments, an antisense oligonucleotide comprises phosphorothioate internucleotide linkages between all nucleotides. For example, in some embodiments, an antisense oligonucleotide comprises modified internucleotide linkages at the first, second, and/or (e.g., and) third internucleotide linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′-alkylene phosphonates and chiral phosphonates, phosphinates, phosphoramidates comprising 3′-amino phosphoramidate and aminoalkylphosphoramidates, thionophos-phoramidates, thionoalkylphosphonates, thionoalkylphosphotriesters, and bora-nophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, the method comprises designing a plurality of ASOs targeting the plurality of high abundance aptamers (e.g., aptamers having a sequence frequency level of higher than 0.1%). In some embodiments, contacting the plurality of ASOs targeting the plurality of high abundance aptamers results in inactivation of the high abundance aptamers such that they are no longer capable of binding to target molecules. In some embodiments, contacting the plurality of ASOs targeting the plurality of high abundance aptamers results in inactivation of the high abundance aptamers such that they are no longer capable of binding to target molecules. In some embodiments, contacting the plurality of ASOs targeting the plurality of high abundance aptamers results in depleting or knocking down the high abundance aptamer in the aptamer library. In some embodiments, contacting the plurality of ASOs targeting the plurality of high abundance aptamers results in depleting or knocking down the high abundance aptamer in the aptamer library by at least 60%, by at least 65%, by at least 70%, by at least 75%, by at least 80%, by at least 85%, by at least 90%, by at least 95%, by at least 96%, by at least 97%, by at least 98%, by at least 99%, or 100%.

In some embodiments, the aptamer library is denatured and renatured to facilitate the formation of the 3D conformation of the aptamers. In some embodiments, denaturing the aptamer library comprises heating the aptamer library for a period of time at a temperature sufficient to break the 3D structure of the aptamers. In some embodiments, denaturing the aptamer library comprises heating the aptamer library for at least 2 minutes, at least 3 minutes, at least 4 minutes, at least 5 minutes, at least 6 minutes, at least 7 minutes, at least 8 minutes, at least 9 minutes, at least 10 minutes, or longer at 90° C., 91° C., 92° C., 93° C., 94° C., 95° C., 96° C., 97° C., 98° C., 99° C., 100° C., or higher. In some embodiments, denaturing the aptamer library comprises heating the aptamer library for 3 minutes at 95° C. In some embodiments, the denatured aptamers are renatured to form the correct 3D conformation. In some embodiments, renaturing the aptamers in the aptamer library comprises cooling down the aptamer library at a temperature for a period of time to a temperature such that the 3D structure of the aptamers are reformed. In some embodiments, the method described herein comprises contacting the ASOs with the aptamer library prior to denaturing of the aptamer library. Contacting the ASOs with the aptamer library prior to denaturing results in stronger knock-down efficiency, but higher non-specific knock down. In some embodiments, the method described herein comprises contacting the ASOs with the aptamer library after renaturing of the aptamer library. Contacting the ASOs with the aptamer library prior to denaturing results in higher specific knock down.

In some embodiments, the method described herein further comprises enriching the plurality of aptamers capable of binding to one or more target molecules in a sample in the following steps: contacting a plurality of candidate aptamers with a sample comprising one or more target molecules to form a composition comprising a plurality of aptamer-target molecule complexes; purifying the plurality of aptamer-target molecule complexes; and extracting the plurality of aptamers capable of binding to one or more target molecules from the aptamer-target molecule complexes.

The term “aptamer-target molecule complex”, as used herein, refers to the molecular complex formed between an aptamer and its target molecule via specific binding. As used herein, the term “specific binding” refers to the ability of a molecule (e.g., an aptamer) to bind to a binding partner (e.g., a target molecule) with a degree of affinity or avidity that enables the molecule to be used to distinguish the binding partner from an appropriate control in a binding assay or other binding context. With respect to an aptamer, the term, “specific binding”, refers to the ability of the aptamer to bind to a target molecule with a degree of affinity or avidity, compared with an appropriate reference target molecule or target molecules, that enables the aptamer to be used to distinguish the specific target molecule from others. In some embodiments, an aptamer specifically binds to a target molecule if the aptamer has a KD for binding the target molecule of at least about 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less.

The term “candidate aptamers”, as used herein, refers to a pool of random aptamers or random oligonucleotides which can be aptamers to be subjected to the methods described herein for enrichment of aptamers capable of binding to one or more target molecules (e.g., biomolecules) from a sample (e.g., a biological sample).

The starting pool of candidate aptamers sequences comprises a random core sequence of 20-60-nt-long species. In some embodiments, the core sequences are flanked by regions that are used for library reamplification (e.g., primer binding site). In some embodiments, it is important to have large diversity in the initial candidate aptamer pool such that the probability of the presence of target-binding aptamers in the initial candidate aptamers.

In some embodiments, in generating an initial plurality of candidate aptamers, the ratio of the nucleotides is optimized, for example, at an A:C:G:T of 1.0:1.0:1.0:1.0, 1.5:1.5:1.0:1.2, 1.30:1.25:1.45:1.00, or 1.50:1.25:1.15:1.00. In some embodiments, a plurality of candidate aptamers comprising at least 10^{14,} at least 10^{15,} at least 10^{16,} at least 10^{17,} at least 10^{18,} at least 10^{19,} at least 10²⁰ or more candidate aptamers.

In some embodiments, the candidate aptamers are an initial pool of random aptamers or random oligonucleotides which can be aptamers that have not being selected. In some embodiments, the candidate aptamers are a resulting plurality of aptamers capable of binding to one or more target molecules (e.g., biomolecules) in a sample (e.g., biological sample) from the last round of enrichment using the method described herein. In some embodiments, the plurality of candidate aptamers are DNA or RNA. In some embodiments, the candidate aptamers are single-stranded DNA (ssDNA). In some embodiments, the plurality of the candidate aptamers are folded in their proper tertiary structure for binding the target molecules. In some embodiments, the plurality of candidate aptamers comprise modified nucleotides. Modified nucleotide have been previously described, see, e.g., Non Patent Literature 12. In some embodiments, each of the plurality of candidate aptamers comprises at least one modified nucleotide. In some embodiments, each plurality of candidate aptamers comprises a modified nucleotide (e.g., modified nucleotide that can be used as a substrate by DNA polymerase). Modified nucleotides are well known in the art (see, e.g., Patent Literature 1, and Non Patent Literature 13 and 14;). In some embodiments, each of the plurality of the candidate aptamers comprises, but are not limited to, for example, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, 2,6-diaminopurine, or 5-tryptamino-uracil. In some embodiments, a modified nucleotide is a 2′-modified nucleotide. For example, the 2′-modified nucleotide may be a 2′-deoxy, 2′-fluoro, 2′-O-methoxyethyl, 2′-amino and 2′-aminoalkoxy modified nucleotides. In some embodiments, each of the plurality of the candidate aptamers comprises but not limited to one or more 5-tryptamino-uracil in place of thymine. In some embodiments, at least one thymine in each of the plurality of the candidate aptamers are replaced by a 5-tryptamino-uracil. In some embodiments, all of the thymine in each of the plurality of the candidate aptamers are replaced by a 5-tryptamino-uracil.

In some embodiments, each of the plurality of the candidate aptamers comprises a detectable label. The detectable label can facilitate detection of aptamer-target molecule (e.g., biomolecule) complexes. In some embodiments, the detectable label is a protein capable of generating a colorimetric change. In some embodiments, the protein capable of generating a colorimetric change is alkaline phosphatase, horseradish peroxidase, or luciferase. In some embodiments, the detectable label is a fluorescent molecule. In some embodiments, the fluorescent molecule includes but are not limited to fluorescent dye is TYE665, lucifer yellow, dansyl, TruRed, fluorescein, Cy2, Cy3, Cy7, TRITC, X-Rhodamine, or Texas red, green fluorescent protein (GFP), blue fluorescent protein (BFP), yellow fluorescent protein (YFP), red fluorescent protein (RFP), mCherry, mTurquoise2, or mOrange. In some embodiments, each of the plurality of the candidate aptamers is labeled at the 5′ end terminal phosphate group. In some embodiments, each of the plurality of the candidate aptamers is labeled at their 3′ end terminal hydroxyl group. In some embodiments, each of the plurality of the candidate aptamers comprises one or more than one detectable label. In some embodiments, each of the plurality of the candidate aptamers comprises a TYE665 fluorophore at the 5′ end.

In some embodiments, prior to contacting the target molecules (e.g., biomolecules) in a sample (e.g., biological sample), the plurality of the candidate aptamers are prepared in a selection buffer. In some embodiments, the selection buffer comprises nonidet-P40. In some embodiments, the selection buffer comprises nonidet-P40 at a con-centration between 0.001% and 0.01%. In some embodiments, the selection buffer comprises nonidet-P40 at a concentration of 0.005%. In some embodiments, the selection buffer comprises a salt (e.g., magnesium ion) capable of stabilization of the tertiary structure of the aptamers. In some embodiments, the selection buffer comprises magnesium ion at a concentration between 0.1 mM and 5 mM, between 0.5 mM and 4.5 mM, between 1 mM and 4 mM, between 2 mM and 3 mM, between 0.5 mM and 2 mM, between 0.6 mM and 1.5 mM, between 0.7 mM and 1.3 mM, between 0.8 mM and 1.2 mM, between 0.9 mM and 1.1 mM, between 0.6 mM and 1.2 mM, between 0.6 mM and 1.2 mM, between 0.7 mM and 1.2 mM, between 0.8 mM and 1.2 mM, between 0.9 mM and 1 mM, between 0.8 mM and 1 mM, between 0.9 mM and 1.5 mM, between 0.9 mM and 1.2 mM, or between 1 mM and 2 mM. In some embodiments, the selection buffer comprises magnesium at a concentration at 1 mM. In some embodiments, the magnesium ion is in the form of magnesium chloride. In some embodiments, the plurality of the candidate aptamers are prepared in the selection buffer at a concentration between 50 nM and 8000 nM, between 60 nM and 7000 nM, between 50 nM and 6000 nM, between 50 nM and 8000 nM, between 50 nM and 8000 nM, between 50 nM and 8000 nM, between 50 nM and 5000 nM, between 60 nM and 4000 nM, between 70 nM and 3000 nM, between 80 nM and 2000 nM, between 90 nM and 1000 nM, between 100 nM and 1000 nM, between 50 nM and 100 nM, between 100 nM and 200 nM, between 200 nM and 500 nM, between 500 nM and 1000 nM, between 1000 nM and 2000 nM, between 2000 nM and 3000 nM, between 3000 nM and 4000 nM, between 4000 nM and 5000 nM, between 1000 nM and 5000 nM, between 1000 nM and 2500 nM, or between 2500 nM and 5000 nM.

In some embodiments, enriching a plurality of aptamers is also described as selecting for a plurality of aptamers. The terms “enriching”, “enrichment”, or “enrichment process” is used interchangeably with selecting, selection, or selection process, respectively. In some embodiments, enriching a plurality of aptamers comprises selection for aptamers that are capable of binding to one or more target molecule (e.g., biomolecules) from those that do not bind to one or more target molecule(s) (e.g., biomolecules). In some embodiments, enriching a plurality of aptamers comprises selection for aptamers with higher affinity to one or more target molecule (e.g., biomolecules) relative to those with lower affinity to one or more target molecule(s) (e.g., biomolecules).

In some embodiments, the method described herein comprises contacting a plurality of candidate aptamers with a sample. In some embodiments, the sample comprises one or more target molecules (e.g., proteins, nucleic acids, toxins, and/or small molecules). In some embodiments, the sample comprises one target molecule. In some embodiments, the sample comprises more than one target molecules.

In some embodiments, the method described herein comprises contacting a plurality of candidate aptamers with a complex sample (e.g., biological sample) comprising a plurality of different target molecules. In some embodiments, a complex sample comprises a plurality of different target molecules of the same type (e.g., the complex sample contains a plurality of different proteins). In some embodiments, a complex sample contains a plurality of different types of target molecules (e.g., the complex sample contains proteins, nucleic acids, small molecules, toxins, etc). In some embodiments, a complex sample contains different types of target molecules and each type of target molecules further contains different individual target molecules (e.g., the complex sample contains a plurality of different proteins, a plurality of different nucleic acids, a plurality of different small molecules, and a plurality of different toxins, etc). Examples of a complex sample include but are not limited to biological sample (e.g., biological fluid such as serum), environmental sample (e.g., samples obtained from river, lake, pond, soil, atmosphere, outer space, etc.), manufacturing sample (e.g., samples obtained from of a bioreactor, samples obtained from a HPLC flow through fluid, samples contain intermediate of a small molecule drug, etc). In some embodiments, a complex sample is a biological sample. The term “biological sample,” as used herein, refers to samples obtained from a biological subject. Examples of biological samples include but are not limited to, whole blood, interstitial fluid, skin, lymphatic fluid, bile, serum, plasma, cerebral-spinal fluid (CSF), urine, amniotic fluid, bone marrow, bronchoalveolar lavage fluid, buccal swab, feces, gastrointestinal fluid, liposuction sample, saliva, milk, nasal swab, peritoneal fluid, semen, sputum, synovial fluid, tears, vaginal fluid, tissue biopsy, autopsy samples, cells or cell lysates, cultured cell, tissue sample (e.g., tissue sample from a human, a non-human animal, plants, insects, fungi), or in vivo endothelial cells. In some embodiments, a biological sample comprises target molecules (e.g., biomolecules) such as nucleic acids (e.g., DNAs and RNAs), proteins, peptides, lipids, polysaccharides, proteoglycans, and glycolipids. In some embodiments, the biological sample is serum. In some embodiments, the biological sample is obtained from a human subject. In some embodiments, the biological sample is obtained from a non-human subject. Examples of non-human subjects include, but are not limited to, monkeys, mice, rats, rabbits, goats, sheep, dogs, birds, and fish. In some embodiments, the subject is a healthy subject. In some embodiments, the subject is a subject suffering, suspected of suffering from a disease, or at a risk of developing a disease.

In some embodiments, the target molecules (e.g., biomolecules) in a sample (e.g., biological sample) are denatured prior to contacting with a plurality of candidate aptamers. In some embodiments, the target molecules (e.g., biomolecules) in a sample (e.g., biological sample) are not denatured prior to contacting with a plurality of candidate aptamers. The term “denature”, “denaturation” or “denatured”, as used herein, refers to subjecting the sample (e.g., biological sample) to a condition that breaks linkages (e.g., disulfide linkages), bonds (e.g., hydrogen bonds, ionic bonds, etc.), and/or interactions (e.g., hydrophobic interactions) within one or more target molecules in the sample (e.g., biomolecules such as proteins, and/or nucleic acids) that are responsible for the highly ordered structure of the target molecule (e.g., biomolecules such as proteins) in its natural (native) state. In some embodiments, a denatured sample comprises a sample wherein the biomolecules (e.g., proteins or nucleic acids) within the sample have lost their quaternary, tertiary, and secondary structure thereby only retaining their primary structure (e.g., linear amino acid sequence or nucleic acid sequence) such that the biomolecules do not retain their respective structures and/or assigned functions. In some embodiments, a denatured sample comprises a sample wherein the biomolecules (e.g., proteins or nucleic acids) have been degraded to fragments such that the biomolecules no longer retain its structure and/or assigned function. As such, a denatured sample comprises biomolecules (e.g., proteins or nucleic acids) that do not retain their assigned function (e.g., binding capabilities, biological activity, etc.). Conversely, a non-denatured sample is one that contains target molecules (e.g., biomolecules) that retain their native conformations and their respective assigned function. Methods for denaturing target molecules in a sample are known in the art, such as by heating, by treatment with alkali, acid, urea, or detergents, or by vigorous shaking. In some embodiments, the target molecules (e.g., biomolecules) are in their native conformation. In some embodiments, contacting a plurality of candidate aptamers with target molecules (e.g., biomolecules) in their native conformation is advantageous in that the selected aptamers are capable of binding to their target molecules (e.g., biomolecules) in other conditions (e.g., in vivo in a subject) than the conditions where the aptamers are selected. It will be understood by those of ordinary skill in the art that denaturation comprises subjecting the target molecules (e.g., biomolecules) in a sample (e.g., biological samples) to a condition (e.g., heating, sonicating, incubating in the presence of a detergent, etc.) sufficient for any change in native or natural structure of a target molecule (e.g. biomolecule such as a protein, DNA, RNA, toxin, or small molecule) that renders it incapable of performing its assigned function. The terms “not denatured”, as used herein, refers to maintaining the structures of the target molecules (e.g., biomolecules) in the sample (e.g., biological samples) sufficient to perform their assigned functions. In some embodiments, the candidate aptamers of the present disclosure can be contacted with a biological sample that is not denatured. In some embodiments, the candidate aptamers of the present disclosure can be contacted with a sample comprising biomolecules (e.g., protein, DNA, RNA, toxin, or small molecule) that retain their quaternary, tertiary, and secondary structures and their respective assigned functions.

The present disclosure recognizes the difficulties of enriching aptamers capable of binding to target molecules in their native confirmation from a complex sample (e.g., a biological sample). The present disclosure also identifies the disadvantage of enriching aptamer-target molecule (e.g., biomolecules) complexes using 1-dimensional electrophoresis (1D-electrophoresis), and/or running electrophoresis under mild conditions (e.g., low salt conditions). For example, and not wishing to be bound to any particular theory, 1D-electrophoresis may not be able to separate aptamer-target molecule complexes from aptamers bound to the target molecules due to non-specific binding. With respect to aptamer, as used herein, the term “non-specific binding”, refers to the ability of the aptamer to bind to a molecule with a degree of affinity or avidity, compared with an appropriate reference target molecule or target molecules, that do not enable the aptamer to be used to distinguish this molecule from others. In some embodiments, an aptamer non-specifically binds to a molecule if the aptamer has a KD for binding the molecule of 10⁻⁶ M, 10⁻⁵ M, 10⁻⁴ M, 10⁻³ M, 10⁻² M, 10⁻¹ M, or higher.

In addition, the condition for 1D-electrophoresis may not be sufficient to break the non-specific binding between certain aptamers and target molecules. The present disclosure, therefore, sought to enriching a plurality of aptamers capable of binding to one or more biomolecule in a biological sample by enriching aptamer-target-molecule complexes by 2D-electrophoresis. The 2D-electrophoresis can be performed under salt conditions sufficient to break the non-specific interaction between an aptamer and a target molecule but not sufficient to break the specific binding between aptamer and its target molecule. Accordingly, the present disclosure enables simultaneous enrichment of a plurality of aptamers capable of binding to multiple target molecules (e.g., biomolecules) in a complex sample (e.g., biological sample)

In some embodiments, the sample (e.g., biological sample) is not diluted prior to contacting with a plurality of candidate aptamers. In some embodiments, the sample (e.g., biological sample) is diluted prior to contacting with a plurality of candidate aptamers. In some embodiments, the biological sample is diluted at a ratio of between 1:1000 and 1:1, between 1:900 and 1:2, between 1:800 and 1:2, between 1:700 and 1:2, between 1:600 and 1:2, between 1:500 and 1:2, between 1:400 and 1:2, between 1:300 and 1:2, between 1:200 and 1:2, between 1:100 and 1:2, between 1:50 and 1:2, between 1:25 and 1:2, between 1:10 and 1:2, between 1:5 and 1:2, between 1:500 and 1:50, between 1:500 and 1:100, between 1:500 and 1:200, between 1:200 and 1:100, between 1:200 and 1:50, between 1:200 and 1:10, between 1:100 and 1:50, between 1:100 and 1:10, between 1:100 and 1:5, or between 1:100 and 1:2 prior to contacting the biological sample with the plurality of candidate aptamers. In some embodiments, the biological sample is a serum, and the serum is diluted at a ratio of between 1:500 and 1:10, between 1:500 and 1:50, between 1:500 and 1:100, between 1:300 and 1:10, between 1:300 and 1:50, between 1:300 and 1:100, between 1:300 and 1:200, between 1:200 and 1:100, between 1:200 and 1:150, between 1:200 and 1:50, between 1:250 and 1:200, or between 1:200 and 1:100, between 1:200 and 1:150, or between 1:200. In some embodiments, the serum is diluted at a ratio of 1;200 prior to contacting the plurality of candidate aptamers. In some embodiments, the biological sample is a CSF, and the CSF is diluted at a ratio of between 1:50 and 1:2, between 1:40 and 1:2, between 1:30 and 1:2, between 1:20 and 1:2, between 1:10 and 1:2, between 1:5 and 1:2, between 1:4 and 1:2, between 1:3 and 1:2, between 1:20 and 1:5, or between 1:10 and 1:5. In some embodiments, the biological sample is diluted in any suitable dilution buffer prior to contacting it with the plurality of candidate aptamers. Non-limiting examples of the dilution buffer include Phosphate-buffered saline (PBS), Dulbecco's phosphate-buffered saline (DPBS), Hanks' Balanced Salt Solution (HBSS), Dulbecco's Modified Eagle Medium (DMEM). One of ordinary skill can select the suitable dilution buffer according to the biological sample being used.

In some embodiments, the method further comprising contacting the sample (e.g., biological sample) to a plurality of competitor nucleic acids prior to contacting the sample (e.g., biological sample) with a plurality of candidate aptamers. The competitor nucleic acids are used to block non-specific binding between aptamers and target molecules. In some embodiments, the competitor nucleic acids are a random set of unrelated nucleic acids. In some embodiments, the competitor nucleic acids are salmon sperm DNA.

In some embodiments, contacting a plurality of candidate aptamers with a sample (e.g., biological sample) results in binding of the aptamers to their target molecules (e.g., biomolecules) to produce a composition. In some embodiments, the composition comprises aptamer-target molecule (e.g., aptamer-biomolecule) complexes. In some embodiments, the composition comprises unbound aptamers. In some embodiments, the composition comprises unbound target molecules (e.g., unbound biomolecules). In some embodiments, the composition comprises aptamer-target molecule (e.g., aptamer-biomolecule) complexes, unbound aptamers, and/or unbound target molecules (e.g., unbound biomolecules). In some embodiments, the composition also comprises aptamers bound to biomolecules due to non-specific binding.

In some embodiments, the method described herein comprises separating the aptamer-target molecule (e.g., aptamer-biomolecule) complexes from the unbound aptamers. In some embodiments, the method described herein comprises separating the aptamer-target molecule (e.g., aptamer-biomolecule) complexes from the unbound aptamers. In some embodiments, the method described herein comprises separating the aptamer-target molecule (e.g., aptamer-biomolecule) complexes from aptamers bound to biomolecules due to non-specific binding. In some embodiments, the present method comprises enriching the aptamer-target molecule (e.g., aptamer-biomolecule complexes) from the composition that also comprises unbound aptamer, and/or aptamers bound to the target-molecules due to non-specific binding.

In some embodiments, enriching the aptamer-target molecule (e.g., aptamer-biomolecule complexes) from the composition that also comprises unbound aptamer, and/or aptamers bound to the target-molecules due to non-specific comprises subjecting the composition to electrophoresis. The term “electrophoresis”, as used herein, refers to a technique used to separate molecules (e.g., DNA, RNA, protein, aptamer-target molecule complexes) based on the size and electrical charge of the molecules being separated. An electrophoretic system includes two electrodes of opposite charge (anode, cathode), connected by a conducting electrophoresis medium. An electric current is used to move molecules to be separated through an electrophoresis medium. In some embodiments, a negatively charged is applied such that the molecules move towards a positive charge, Further, the electrophoresis medium usually contains pores that allow smaller molecules to move faster than larger molecules. In some embodiments, the present disclosure is based on the theory that the plurality of aptamer-target molecules (e.g., biomolecule) complexes are different in size from the unbound aptamer (e.g., biomolecules). In some embodiments, the unbound aptamers are smaller in size than the aptamer-target molecule (e.g., biomolecule) therefore migrate faster during electrophoresis. In some embodiments, the individual aptamer-target molecule (e.g., biomolecule) in the plurality of aptamer-target molecules (e.g., biomolecule) complexes are different in size from each other. In some embodiments, the individual aptamer-target molecule (e.g., biomolecule) in the plurality of aptamer-target molecule (e.g., biomolecule) complexes migrate at different speed during electrophoresis. In some embodiments, the methods described herein comprises subjecting the composition to electrophoresis in a first electrophoresis medium in a first direction. In some embodiments, the size difference between aptamer-target molecule (e.g., biomolecule) complexes and aptamers bound to the target molecules (e.g., biomolecules) due to non-specific binding is not sufficient to separate the aptamer-target molecule (e.g., biomolecule) complexes from the aptamers bound to the target molecules (e.g., biomolecules) due to non-specific binding. In some embodiments, after the electrophoresis in the first direction, a portion of the first electrophoresis medium comprises the aptamer-target molecule complexes, and the same portion may also contain the aptamers bound to the target molecules (e.g., biomolecules) due to non-specific binding.

Accordingly, in some embodiments, the present disclosure sought to enriching a plurality of aptamers capable of binding to one or more biomolecule in a biological sample by enriching aptamer-target molecule (e.g., biomolecule) complexes by 2D-electrophoresis. In some embodiments, the method comprising subjecting the portion of the first electrophoresis medium that comprises the aptamer-target molecule complexes, which may also contain aptamers bound to the target molecules (e.g., biomolecules) due to non-specific binding to electrophoresis in a second electrophoresis medium in a second direction. The term “second direction” as used herein, refers to a direction different from the first direction. In some embodiments, a second direction is orthogonal to the first direction. In some embodiments, the method further comprising excising the portion of the first electrophoresis medium comprising the aptamer-target molecule complexes (which may also contain aptamers bound to the target molecules (e.g., biomolecules) due to non-specific binding) from the rest of the first electrophoresis medium. In some embodiments, the method further comprises fit the excised portion of the first electrophoresis medium comprising the aptamer-target molecule complexes (which may also contain aptamers bound to the target molecules (e.g., biomolecules) due to non-specific binding) in a well in the second electrophoresis medium. Subjecting the composition to electrophoresis in a second dimension breaks the weak, non-specific binding between aptamers and the target molecules (e.g., biomolecules), such that these aptamers become unbound aptamers and migrate faster than the aptamer-target molecule (e.g., biomolecule) complexes. In some embodiments, the aptamer-target molecule (e.g., biomolecule) complexes presents in the second electrophoresis medium in the diagonal region after electrophoresis in the second direction. In some embodiments, the method further comprising excising the portion of the second electrophoresis medium comprising the aptamer-target molecule complexes from the rest of the second electrophoresis medium.

In some embodiments, the method further comprises extracting the aptamers capable of binding to one or more target molecules (e.g., biomolecules) from the portion of the second electrophoresis medium that contains the aptamer-target molecule complexes. In some embodiments, the aptamers are separated from the aptamer-target molecule complexes during this step. Methods of extracting the aptamers from a portion of an electrophoresis medium has been previously described, e.g., by a commercially available kit such as Oligo Clean and Concentrator Kits by Zymo Research), QIAquick Gel Extraction Kit (QIAGEN, Hilden, Germany), Wizard SV Gel and PCR Clean Up System (Promega, Madison, WI, USA), and GENECLEAN (Registered Trademark) II kit (MP BIOMEDICALS, Solon, OH, USA), or by the method described in Non Patent Literature 15.

In some embodiments, an electrophoresis medium comprises pores allowing migration of the molecules being subjected to electrophoresis. Non-limiting examples of electrophoresis medium includes agarose, polyacrylamide, silica matrix, or starch. In some embodiments, the first electrophoresis medium is agarose. In some embodiments, the second electrophoresis medium is agarose. In some embodiments, the first electrophoresis medium is agarose, and the second electrophoresis medium is agarose. Methods of preparing an agarose gel for electrophoresis are known in the art, e.g., as described by Non Patent Literature 16. In some embodiments, the agarose gel is prepared from dry agarose power (e.g., by dissolving the agarose power in a suitable buffer such as Tris Buffer by heating, and letting the agarose solidify by cooling to room temperature). In some embodiments, the agarose gel is a pre-made gel purchased from a vendor. In some embodiments, the first electrophoresis medium is an agarose gel and comprises agarose at a concentration of any concentration between 0.5% and 3% (e.g., 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5%), between 1%-2%, between 0.5%-1%, between 1.5%-2%, or between 2%-2.5%.

In some aspects, the present disclosure also contemplates the optimal conditions (e.g., salt, temperature) for performing the electrophoresis. In some embodiments, the 2D-electrophoresis is performed under a salt condition (e.g., the electrophoresis medium comprises an ion at a concentration) capable of mitigating electrostatic effect of biomolecules (e.g., proteins or DNAs). In some embodiments, the 2D-electrophoresis is performed under salt conditions (e.g., the electrophoresis medium comprises an ion at a concentration) sufficient to break the non-specific interaction between an aptamer and a target molecule but not sufficient to break the specific binding between aptamer and its target molecule. In some embodiments, the first electrophoresis medium comprises sodium ion (e.g., sodium chloride), potassium ion, lithium ion, ammonium ion or any combination thereof at a concentration sufficient to break non-specific binding of aptamers to target-molecules. In some embodiments, such ion includes but are not limited to sodium ion, potassium ion, lithium ion, ammonium ion, or any combination thereof (e.g., combination of sodium ion and potassium ion; combination of sodium ion and lithium ion; combination of sodium ion and ammonium ion; combination of potassium ion and lithium ion; combination of potassium ion and ammonium ion; combination of lithium ion and ammonium ion; combination of sodium ion, potassium ion, and lithium ion; combination of sodium ion, potassium ion, and ammonium ion; combination of potassium ion, lithium ion, and ammonium ion; or combination of sodium ion, potassium ion, lithium ion, and ammonium ion). The term “any combination thereof at a concentration”, as used herein, refers to a total concentration of the ion(s) in a combination (e.g., any of the combination described herein). For example, a combination of two ions at a concentration between X and Y refers to a total concentration of the two ions is within the range of X and Y; a combination of three ions at a concentration of between X and Y refers to a total concentration of the three ions is within the range of X and Y, etc. A concentration of can be described using any unit known in the art, e.g., M, mM, μM, nM, pM, g/L, g/dL, g/mL, g/μL, g/nL, mg/L, mg/dL, mg/mL, mg/μL, mg/nL, μg/L, μg/dL, μg/mL, μg/μL, μg/nL, ng/L, ng/dL, ng/ml, ng/μL, ng/nL, pg/L, pg/dL, pg/mL, pg/μL, or pg/nL. For example, when an electrophoresis medium comprises a combination of ions (e.g., sodium, potassium, lithium, and/or ammonium ions) at a total concentration between 100 mM and 200 mM, it means that the total concentration of the ions thereof is between 100 mM and 200 mM. It is within the skill of one of ordinary skill in the art to select a concentration for each ion in the combination to reach a total concentration of a prescribed range.

In some embodiments, the first electrophoresis medium comprises sodium ion (e.g., sodium chloride), potassium ion, lithium ion, ammonium ion or any combination thereof at a concentration between 50 mM and 500 mM, between 80 mM and 450 mM, between 100 mM and 400 mM, between 150 mM and 350 mM, between 200 mM and 300 mM, between 100 mM and 400 mM, between 100 mM and 300 mM, between 100 mM and 200 mM, between 100 mM and 150 mM, between 150 mM and 200 mM, between 110 mM and 190 mM, between 120 mM and 180 mM, between 130 mM and 170 mM, between 140 mM and 160 mM, between 120 mM and 150 mM, between 120 mM and 160 mM, between 120 mM and 170 mM, between 120 mM and 130 mM, between 150 mM and 160 mM, between 150 mM and 170 mM, between 150 mM and 180 mM, between 150 mM and 190 mM, between 100 mM and 120 mM, between 120 mM and 130 mM, between 130 mM and 140 mM, between 140 mM and 150 mM, between 150 mM and 160 mM, between 160 mM and 170 mM, between 170 mM and 180 mM, between 180 mM and 190 mM, or between 190 mM and 200 mM. In some embodiments, the first electrophoresis medium comprises sodium ion (e.g., sodium chloride), potassium ion, lithium ion, ammonium ion or any combination thereof at a concentration between 100 mM and 200 mM (e.g., any concentration between 100 mM and 200 mM). In some embodiments, the second electrophoresis medium comprises sodium chloride at a concentration between 50 mM and 500 mM, between 80 mM and 450 mM, between 100 mM and 400 mM, between 150 mM and 350 mM, between 200 mM and 300 mM, between 100 mM and 400 mM, between 100 mM and 300 mM, between 100 mM and 200 mM, between 100 mM and 150 mM, between 150 mM and 200 mM, between 110 mM and 190 mM, between 120 mM and 180 mM, between 130 mM and 170 mM, between 140 mM and 160 mM, between 120 mM and 150 mM, between 120 mM and 160 mM, between 120 mM and 170 mM, between 120 mM and 130 mM, between 150 mM and 160 mM, between 150 mM and 170 mM, between 150 mM and 180 mM, between 150 mM and 190 mM, between 100 mM and 120 mM, between 120 mM and 130 mM, between 130 mM and 140 mM, between 140 mM and 150 mM, between 150 mM and 160 mM, between 160 mM and 170 mM, between 170 mM and 180 mM, between 180 mM and 190 mM, or between 190 mM and 200 mM. In some embodiments, the second electrophoresis medium comprises sodium ion (e.g., sodium chloride), potassium ion, lithium ion, ammonium ion or any combination thereof at a concentration between 100 mM and 200 mM (e.g., any concentration between 100 mM and 200 mM).

In addition, and in some embodiments, the 2D-electrophoresis is performed under salt conditions (e.g., a divalent ion at a concentration) sufficient to stabilize the structure of aptamers and/or the aptamer-target molecule (e.g., biomolecule) complexes. Non-limiting examples of divalent ion capable of stabilizing the structure of aptamers and/or the aptamer-target molecule (e.g., biomolecule) complexes include but are not limited to magnesium ion, calcium ion, copper ion, zinc ion, or any combination thereof. In some embodiments, the first electrophoresis medium comprises magnesium ion, calcium ion, copper ion, zinc ion or any combination thereof at a concentration sufficient to stabilize the structure of aptamers and/or the aptamer-target molecule (e.g., biomolecule) complexes. In some embodiments, the first electrophoresis medium comprises magnesium ion, calcium ion, copper ion, zinc ion or any combination thereof at a concentration of 10 mM or less, (e.g., less than 10 mM, less than 9 mM, less than 8 mM, less than 7 mM, less than 6 mM, less than 5 mM, less than 4 mM, less than 3 mM, less than 2 mM, less than 1 mM, or less than 0.5 mM). In some embodiments, the first electrophoresis medium comprises magnesium ion, calcium ion, copper ion, zinc ion or any combination thereof at a concentration of between 0.1 mM and 10 mM, between 0.1 mM and 9 mM, between 0.1 mM and 8 mM, between 0.1 mM and 7 mM, between 0.1 mM and 6 mM, between 0.1 mM and 4 mM, between 0.1 mM and 3 mM, between 0.1 mM and 2 mM, between 0.1 mM and 1 mM, between 0.1 mM and 0.5 mM, between 0.5 mM and 10 mM, between 0.5 mM and 9 mM, between 0.5 mM and 8 mM, between 0.5 mM and 7 mM, between 0.5 mM and 6 mM, between 0.5 mM and 5 mM, between 0.5 mM and 4 mM, between 0.5 mM and 3 mM, between 0.5 mM and 2 mM, between 0.5 mM and 1 mM, between 1 mM and 10 mM, between 1 mM and 9 mM, between 1 mM and 8 mM, between 1 mM and 7 mM, between 1 mM and 6 mM, between 1 mM and 5 mM, between 1 mM and 3 mM, between 1 mM and 2 mM, between 2 mM and 10 mM, between 2 mM and 9 mM, between 2 mM and 8 mM, between 2 mM and 7 mM, between 2 mM and 6 mM, between 2 mM and 5 mM, between 2 mM and 4 mM, between 3 mM and 10 mM, between 3 mM and 9 mM, between 3 mM and 8 mM, between 3 mM and 7 mM, between 3 mM and 6 mM, between 3 mM and 5 mM, between 3 mM and 4 mM, between 4 mM and 10 mM, between 4 mM and 9 mM, between 4 mM and 8 mM, between 4 mM and 7 mM, between 4 mM and 6 mM, between 4 mM and 5 mM, between 5 mM and 10 mM, between 5 mM and 9 mM, between 5 mM and 8 mM, between 5 mM and 7 mM, between 5 mM and 6 mM, between 6 mM and 10 mM, between 6 mM and 9 mM, between 6 mM and 8 mM, between 6 mM and 7 mM, between 7 mM and 10 mM, between 7 mM and 9 mM, between 7 mM and 8 mM, between 8 mM and 10 mM, between 8 mM and 9 mM, between 8 mM and 9 mM, between 0.1 mM and 5 mM, between 0.5 mM and 4.5 mM, between 1 mM and 4 mM, between 2 mM and 3 mM, between 0.5 mM and 2 mM, between 0.6 mM and 1.5 mM, between 0.7 mM and 1.3 mM, between 0.8 mM and 1.2 mM, between 0.9 mM and 1.1 mM, between 0.6 mM and 1.2 mM, between 0.6 mM and 1.2 mM, between 0.7 mM and 1.2 mM, between 0.8 mM and 1.2 mM, between 0.9 mM and 1 mM, between 0.8 mM and 1 mM, between 0.9 mM and 1.5 mM, between 0.9 mM and 1.2 mM, or between 1 mM and 2 mM. In some embodiments, the first electrophoresis medium comprises magnesium ion, calcium ion, copper ion, zinc ion or any combination thereof at a concentration at 1 mM. In some embodiments, the second electrophoresis medium comprises magnesium ion, calcium ion, copper ion, zinc ion or any combination thereof at a concentration sufficient to stabilize the aptamer-target molecule (e.g., biomolecule) complexes. In some embodiments, the second electrophoresis medium comprises magnesium ion, calcium ion, copper ion, zinc ion or any combination thereof at a concentration of 10 mM or less, (e.g., less than 10 mM, less than 9 mM, less than 8 mM, less than 7 mM, less than 6 mM, less than 5 mM, less than 4 mM, less than 3 mM, less than 2 mM, less than 1 mM, or less than 0.5 mM). In some embodiments, the first electrophoresis medium comprises magnesium ion, calcium ion, copper ion, zinc ion or any combination thereof at a concentration of between 0.1 mM and 10 mM, between 0.1 mM and 9 mM, between 0.1 mM and 8 mM, between 0.1 mM and 7 mM, between 0.1 mM and 6 mM, between 0.1 mM and 4 mM, between 0.1 mM and 3 mM, between 0.1 mM and 2 mM, between 0.1 mM and 1 mM, between 0.1 mM and 0.5 mM, between 0.5 mM and 10 mM, between 0.5 mM and 9 mM, between 0.5 mM and 8 mM, between 0.5 mM and 7 mM, between 0.5 mM and 6 mM, between 0.5 mM and 5 mM, between 0.5 mM and 4 mM, between 0.5 mM and 3 mM, between 0.5 mM and 2 mM, between 0.5 mM and 1 mM, between 1 mM and 10 mM, between 1 mM and 9 mM, between 1 mM and 8 mM, between 1 mM and 7 mM, between 1 mM and 6 mM, between 1 mM and 5 mM, between 1 mM and 3 mM, between 1 mM and 2 mM, between 2 mM and 10 mM, between 2 mM and 9 mM, between 2 mM and 8 mM, between 2 mM and 7 mM, between 2 mM and 6 mM, between 2 mM and 5 mM, between 2 mM and 4 mM, between 3 mM and 10 mM, between 3 mM and 9 mM, between 3 mM and 8 mM, between 3 mM and 7 mM, between 3 mM and 6 mM, between 3 mM and 5 mM, between 3 mM and 4 mM, between 4 mM and 10 mM, between 4 mM and 9 mM, between 4 mM and 8 mM, between 4 mM and 7 mM, between 4 mM and 6 mM, between 4 mM and 5 mM, between 5 mM and 10 mM, between 5 mM and 9 mM, between 5 mM and 8 mM, between 5 mM and 7 mM, between 5 mM and 6 mM, between 6 mM and 10 mM, between 6 mM and 9 mM, between 6 mM and 8 mM, between 6 mM and 7 mM, between 7 mM and 10 mM, between 7 mM and 9 mM, between 7 mM and 8 mM, between 8 mM and 10 mM, between 8 mM and 9 mM, between 8 mM and 9 mM, between 0.1 mM and 5 mM, between 0.5 mM and 4.5 mM, between 1 mM and 4 mM, between 2 mM and 3 mM, between 0.5 mM and 2 mM, between 0.6 mM and 1.5 mM, between 0.7 mM and 1.3 mM, between 0.8 mM and 1.2 mM, between 0.9 mM and 1.1 mM, between 0.6 mM and 1.2 mM, between 0.6 mM and 1.2 mM, between 0.7 mM and 1.2 mM, between 0.8 mM and 1.2 mM, between 0.9 mM and 1 mM, between 0.8 mM and 1 mM, between 0.9 mM and 1.5 mM, between 0.9 mM and 1.2 mM, or between 1 mM and 2 mM. In some embodiments, the second electrophoresis medium comprises magnesium ion, calcium ion, copper ion, zinc ion or any combination thereof at a concentration at 1 mM. Magnesium can be added to the first and/or the second electrophoresis medium in any known magnesium salt from such as magnesium chloride, and magnesium sulfate. In some embodiments, the magnesium ion is in the form of magnesium chloride.

In some embodiments, the running buffer of the electrophoresis in the first direction comprises boric acid at a concentration between 40 mM and 100 mM. In some embodiments, the running buffer of the electrophoresis in the first direction comprises tris(hydroxymethyl)aminomethane at a concentration between 40 mM and 100 mM. In some embodiments, the running buffer of the electrophoresis in the second direction comprises boric acid at a concentration between 40 mM and 100 mM. In some embodiments, the running buffer of the electrophoresis in the second direction comprises tris(hydroxymethyl)aminomethane at a concentration between 40 mM and 100 mM.

In some embodiments, the 2D-electrophoresis is performed at a temperature optimal for migration and separation of the molecules (e.g., aptamer-biomolecule complexes, and unbound aptamer) in the composition. In some embodiments, the electrophoresis in the first direction is performed at a temperature between 8° C. and 22° C., between 9° C. and 21°° C., or between 10° C. and 20° C., between 11° C. and 19° C., between 12° C. and 18° C., between 13° C. and 17° C., between 14° C. and 16° C., between 10° C. and 15° C., between 11° C. and 14° C., between 12° C. and 13° C. In some embodiments, the electrophoresis in the first direction is performed at 8° C., 9° C., 10° C., 11° C., 12° C., 13° C., 14° C., 15° C., 16° C., 17° C., 18° C., 19° C., 20° C., 21° C., or 22° C. In some embodiments, the electrophoresis in the second direction is performed at a temperature between 8° C. and 22° C., between 9° C. and 21° C., or between 10° C. and 20° C., between 11° C. and 19°° C., between 12° C. and 18° C., between 13° C. and 17° C., between 14° C. and 16° C., between 10° C. and 15° C., between 11° C. and 14° C., between 12° C. and 13° C. In some embodiments, the electrophoresis in the second direction is performed at 10° C., 11° C., 12° C., 13° C., 14° C., 15° C., 16° C., 17° C., 18° C., 19° C., or 20° C. In some embodiments, the amount of the salt (e.g., sodium chloride and/or magnesium chloride) in the first and the second electrophoresis medium increases the temperature of the electrophoresis medium to a temperature that may interfere with the stability of the aptamer-target molecule complexes. In some embodiments, in order to keep the temperature at the optimal temperature range described herein, the electrophoresis unit is placed in a cold water bath filled with ice.

As described herein, in some embodiments, the method further comprises extracting the aptamers capable of binding to one or more target molecules (e.g., biomolecules) from the portion of the second electrophoresis medium that contains the aptamer-target molecule complexes. In some embodiments, the method further comprising amplifying the plurality of aptamers extracted from the aptamer-target molecule (e.g., biomolecule) complexes to form an aptamer library. In some embodiments, amplification of the aptamers capable of binding to one or more target molecules (e.g., biomolecules) is by polymerase chain reaction (PCR). Polymerase chain reaction (PCR) is a laboratory technique used to amplify DNA sequences by using short DNA sequences called primers. The temperature of the sample is repeatedly raised and lowered to help a DNA replication enzyme copy the target DNA sequences. Non-limiting examples of PCR includes emulsion PCR, Asymmetric PCR, Convective PCR, Dial-out PCR, Digital PCR, Helicase-dependent amplification, Hot start PCR, in silico PCR, Inverse PCR, Ligation-mediated PCR, Miniprimer PCR, Multiplex ligation-dependent probe amplification, Multiplex-PCR, Nanoparticle-Assisted PCR, Nested PCR, Overlap-extension PCR, quantitative PCR, Reverse Complement PCR, Single Specific Primer-PCR, and Solid Phase PCR. In some embodiments, the aptamers are amplified by emulsion PCR.

In some embodiments, the plurality of aptamers capable of binding to one or more target molecules (e.g., biomolecules) extracted from the portion of the second electrophoresis medium that contains the aptamer-target molecule complexes comprises aptamers in different amounts (i.e., a sequence order), e.g., aptamers binding to target molecules with higher affinity are present at a higher level whereas aptamers binding to target molecules with lower affinity are present at a lower level. In some aspects, the present disclosure sought to preserve the sequence order information in the plurality of aptamers capable of binding to one or more target molecules (e.g., biomolecules) after forming the aptamer library by amplification. In some embodiments, the present disclosure is based on the discovery that emulsion PCR is capable of preserving the sequence order information in the plurality of aptamers capable of binding to one or more target molecules (e.g., biomolecules) after forming the aptamer library by amplification.

In some embodiments, sequencing the low abundance aptamers in an aptamer library is performed through multiple rounds of selecting for aptamers capable of binding to one or more target molecules (e.g., biomolecules) in a sample (e.g., biological sample), amplifying the aptamers capable of binding to the target molecules by emulsion PCR to form the aptamer library, sequencing the aptamer library; and knocking down the high abundance aptamer using ASOs. In some embodiments, the steps of the methods described herein are repeated at least once, at least twice, at least three times, at least four times, at least five times, at least six times, at least seven times, at least eight times, at least nine times, at least ten times, at least eleven times, at least twelve times, at least thirteen times, at least fourteen times, at least fifteen times, or more times. In some embodiments, the steps of the methods described herein are repeated four times.

In some embodiments, the steps of the methods described herein are repeated nine times. In some embodiments, the aptamer library obtained after amplification (e.g., emulsion PCR) is used as the starting plurality of candidate aptamers for contacting the target molecules (e.g., biomolecules) in the sample (e.g., biological sample) in the next round.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an”, as used herein in the specification and in the claims, unless clearly indicated to the contrary, should be understood to mean “at least one.”

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of”, “only one of”, or “exactly one of”. “Consisting essentially of”, when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B”, or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

### EXAMPLES

### Example 1: Materials and Methods

### Antisense Oligonucleotide Design

Antisense oligonucleotide (ASO) was systematically designed based on an aptamer sequence information that was obtained by next generation sequencing analysis (NGS) after SELEX. The length of ASO was fixed at 25-30 nucleotides for all ASOs. Target domain started from position 7-10 of 5′-end of random region. Target sequences to be knocked down by ASOs were determined based on sequence frequency level to be over 0.1-0.2% to maximize available sequencing reads for remaining sequences after ASOs-based depletion. All ASOs are composed of DNA.

### Interaction of Antisense Oligonucleotides With Aptamer Library

ASOs were dissolved in deionized water before use. The working concentration of ASOs was set at 100-1000 times higher than target sequence concentration that can be calculated from NGS data of an aptamer library. A 50× ASO solution was prepared by mixing ASOs in a tube. The targeting nucleic library prepared by the method of 2D-electrophoresis-based aptamer library generation was diluted in the selection buffer (PBS with 0.005% nonidet-P40 and 1 mM magnesium chloride) at the working concentration for selection. The 50× ASO solution was added to the aptamer library solution before denaturation of the library for eliciting strong knock-down efficiency. The solution was denatured at 95° C. for 3 min then slowly cooled down to room temperature over 30 min to form stable ssDNA structures. At this step, ASO-targeted aptamers are inactivated due to inhibition of functional structure formation. If target specificity is more important than knock-down efficiency, the 50× ASO solution was added after the renaturation step. The renatured library solution was mixed with an appropriately diluted biofluid sample solution at 10 μL reaction volume. The biofluid sample was mixed with competitors, such as salmon sperm DNA and any unrelated oligo DNAs, in advance to mixing with the ssDNA library solution. Dextran sulfate was also employed at a concentration ranging from 0.001% to 1% to reduce charge-dependent nonspecific interaction of ssDNA with biomolecules. The mixture was incubated at room temperature for 10 min, and then 2 μL of 6× loading buffer comprising 36% glycerol and 6 mM magnesium chloride was added to the reaction mixture quickly followed by the sample load on the agarose gel.

### Preparation of Agarose Gel for 1D- and 2D-Electrophoresis

The agarose gels for 1D- and 2D-electrophoresis were prepared as follows. Agarose powder was mixed at a concentration of 1.0-1.2 wt % in 1× TB buffer (80 mM tris base, 80 mM boric acid) including 100-200 mM NaCl concentration depending on the selection condition. The mixture was heated by microwave to completely dissolve agarose powder. Then, after cooling the agarose solution to around 60° C., 1 M MgCl₂ solution was added to a concentration of 1 mM MgCl₂ and mixed thoroughly. Then, 11 mLs of the solution was poured into a gel tray (55 mm(W)×60 mm(L)) with a well comb in place. The number of wells was 3-4 wells (10 mm(W)×1 mm(L)) for 1D-electrophoresis and 1 well (45 mm(W)×2 mm(L)) for 2D-electrophoresis. The thickness of agarose gel was adjusted to be relatively thin to decrease heat generated from high-salt agarose gel during electrophoresis. The gel was placed at room temperature for at least 3 hours, but less than 6 hours, before use.

### Removal of Targeted Aptamer Clusters From Library by 2D-Electrophoresis

An electrophoresis unit, such as Mupid2, was filled with 1× TB buffer. The gel for 1D-electrophoresis prepared above was placed in the gel box and the electrophoresis unit was placed in a cold water bath filled with crushed ice to keep the buffer temperature between 10-20° C. during electrophoresis due to the use of high-salt agarose gel. The gel was pre-run at 100 V for 10 min before sample run. The sample mixture was carefully loaded on the well of the gel. First-dimensional electrophoresis was performed at 100 V for 55-60 min in the dark place. After the run, the gel was taken from the gel box and visualized by ChemiDoc MP Imaging System (Bio-Rad). The entire region of aptamer-biomolecule complexes that are generally present above free aptamer region was excised for 2D-electrophoresis. The size of excised gel was adjusted to the well of 2D-electrophoresis gel.

As with 1D-electrophoresis, 2D-electrophoresis was performed. After the run, the gel was taken from the gel box for visualization by ChemiDoc MP Imaging System (Bio-Rad) and only diagonal region formed by aptamer-biomolecule complexes was excised. The excised gel piece was placed in a microtube.

### DNA Extraction From Agarose Gel

The excised gel piece was melted at 95° C. for 3 min. The melted agarose solution was distributed into several tubes with 110 μL aliquots. The tubes were incubated at 55° C. for 1 min. Thermostable β-agarase was added to each tube by 2 μL and the tubes were incubated at 55° C. for 15 min to enzymatically digest agarose. The ssDNA library was recovered by Oligo Clean and Concentrator Kits (Zymo Research), following manufacturer's instructions. The recovered ssDNA was eluted with 30 μL of deionized water.

### Emulsion PCR Amplification of Recovered ssDNA Library

The recovered ssDNA was subjected to emulsion PCR amplification in a two-step process. Note that all PCR reactions should be performed by emulsion PCR to keep sequence order information in the library after removal of high frequency sequences. Emulsion PCR solution was prepared as follows: 100 μL of PCR solution was mixed with 250 μL of emulsion oil (4.5% Span80, 0.4% Tween 80, 0.05% Triton-X 100, and 95.05% Mineral Oil) and the solution was vigorously mixed by magnetic stir bar until completely mixed. Emulsion PCR reaction was tested by different numbers of sequential PCR cycles with a small volume. The PCR products at defined cycles were recovered by chloroform extraction and analyzed by gel electrophoresis (6% poly-acrylamide with 0.5× TBE buffer) to determine a proper PCR cycle that can provide a clear single band for DNA products without any concatemers and truncations. The remaining PCR sample was amplified by employing the determined PCR cycle. The PCR products were recovered by chloroform extraction and subjected to purification by Oligo Clean and Concentrator Kits. The amplified DNA was eluted with 10 μL of deionized water and quantified by Qubit 4 Fluorometer (Invitrogen). This PCR amplification step was repeated until enough amount of DNA was obtained.

### Aptamer Library Generation by Primer Extension

In some cases, 2 to 3 cycles are necessary to sufficiently remove target sequences from the library. The amplified DNA was diluted to the concentration of 1 ng/μL to prepare template DNA sample for aptamer library generation by primer extension. Then 2 μL of the solution were used for 100 μL of each PCR amplification. In this PCR amplification step, 5′-biotinylated antisense strand primer was employed, and the number of PCR cycle was fixed to 8 cycles. The PCR products were recovered by chloroform extraction followed by Oligo Clean and Concentrator Kits purification. The purified DNA was subjected to primer extension by using 5′-TYE665 fluorophore-labeled primer. The products were directly immobilized on streptavidin agarose beads filled with PBSN buffer (PBS with 0.005% nonidet-P40). The beads were incubated at 16° C. with shaking at 1500 rpm every 2 min for 15 min. The beads were washed three times with PBS and filled with 40 μL of 20 mM NaOH solution for denaturation of the product DNA to dissociate 5′-TYE665-labeled aptamers. The beads solution was incubated at 37° C. with shake at 1500 rpm for 1 min. The solution was centrifuged by a benchtop mini centrifuge for 30 sec. The supernatant was transferred to a new tube. The denaturation step was performed once more, and the supernatants were combined in the same tube. The collected supernatant was quenched by 80 mM HCl to adjust the solution pH around 7.0-8.0 and purified by Oligo Clean and Concentrator Kits. The aptamer library was eluted with 10 μL of deionized water and quantified by NanoDrop. The aptamer library was used for the next cycle of selection.

### Sequencing Analysis of Aptamer Library

The PCR products obtained after 2D-electrophoresis were emulsion PCR amplified in a 2-step process to prepare for next generation sequencing (NGS) analysis by MiSeq (illumina). Adapter sequence primer set was used in the first step followed by using index sequence primer set in the second step. Thermal cycling was performed as in the manufacturer's instruction. The PCR products were purified by NucleoSpin Gel and PCR Clean-up (Macherey Nagel) and quantified by Qubit 4 fluorometer for the first step and Bioanalyzer for the second step. The products were analyzed by MiSeq following manufacturer's instructions. All sequencing data for each round were generated as FASTAQ files. After extracting aptamer domain by trimming 5′- and 3′-primer regions, all sequences were used for frequency analysis.

### Example 2: ASO Depletion of High Abundance Aptamers in an Aptamer Library

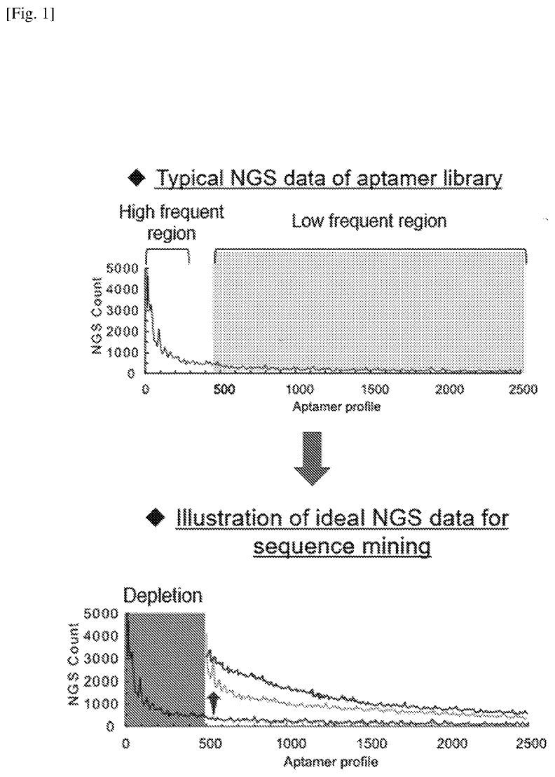

This example describes methods for knocking down of high abundance aptamers in a library of aptamers. In an aptamer library, high abundance aptamers (e.g., aptamers with higher affinity to a target molecule) comprise a higher number of NGS reads relative to low abundance aptamers (e.g., aptamers with lower affinity to a target molecule). In turn, low abundance aptamers exhibit lower detectability by NGS analysis, i.e., although aptamers in the low frequent region are difficult to analyze due to lower reads, more sequences should exist under the region of detection limit. When high frequency aptamers were depleted from the library of aptamers detectability of low abundance aptamers capable of binding to target biomolecules was increased, i.e., Depletion of high frequent sequences resulted in increase of low frequent sequences (FIG. 1).

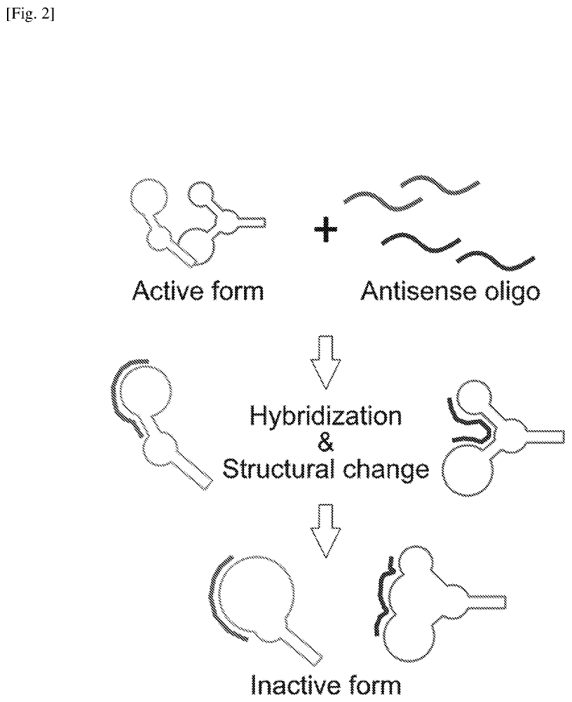

This method further describes depleting high abundance aptamers from an aptamer library by ASOs. ASOs are designed to target high abundance aptamers (e.g., aptamers with percentage reads over a certain threshold). Hybridization of ASOs to high abundance aptamers induced structural changes that inactivates binding capabilities of high abundance aptamers to target molecules (FIG. 2).

### Example 3: Manipulation of Aptamer Library for Mouse Serum

The method according to the present disclosure was applied for the manipulation of aptamer library generated for mouse serum.

The aptamer library was generated and sequenced based on the method for generating aptamer library using an unpurified biological sample that were developed separately.

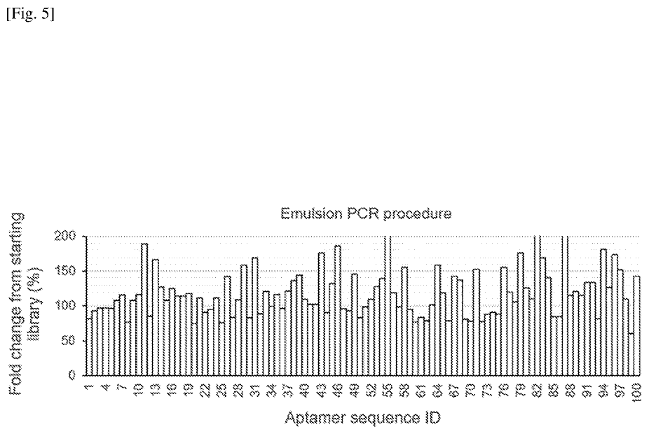

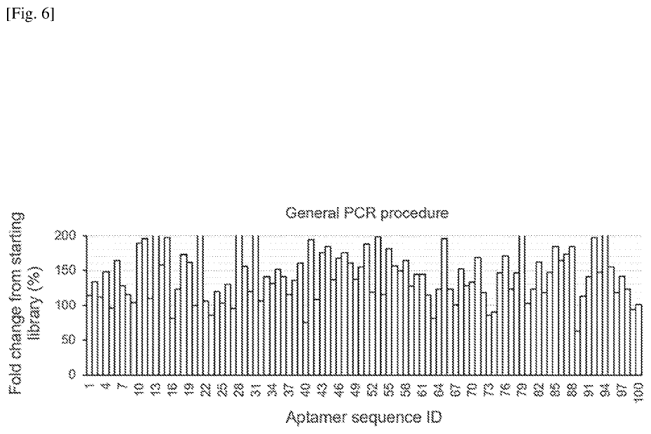

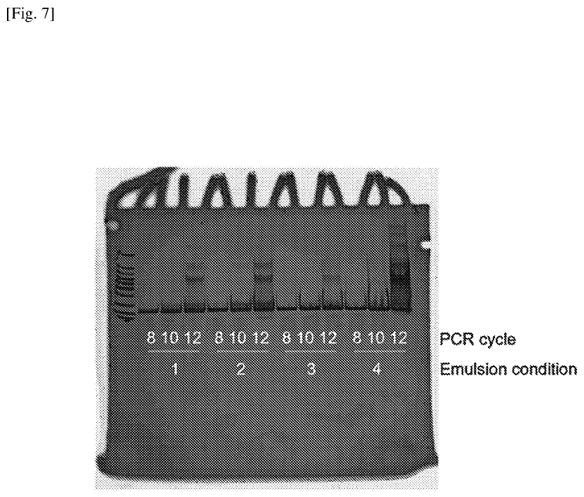

According to the present disclosure, the emulsion PCR was used to keep sequence order information after depletion of targeted sequences. FIGS. 5 and 6 show exemplary data that compared the PCR product between emulsion PCR and general PCR methods obtained by duplicating the same aptamer library at the same time. FIG. 7 shows data for PCR product check by PAGE. Emulsion conditions are as follows: 1; combination of sonication and voltex, 2; voltex, 3; string by magnetic stirrer bar, 4; general PCR.

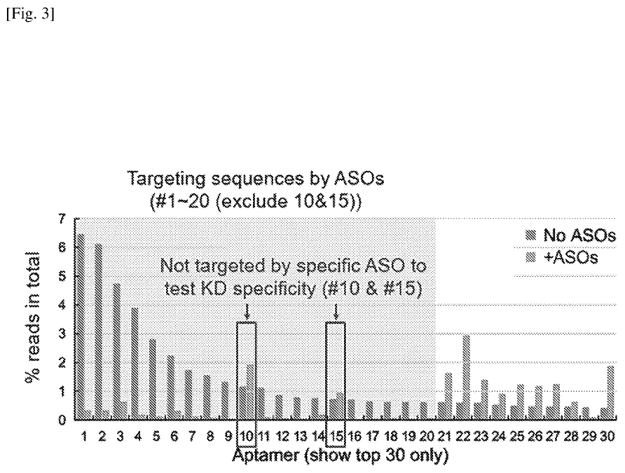

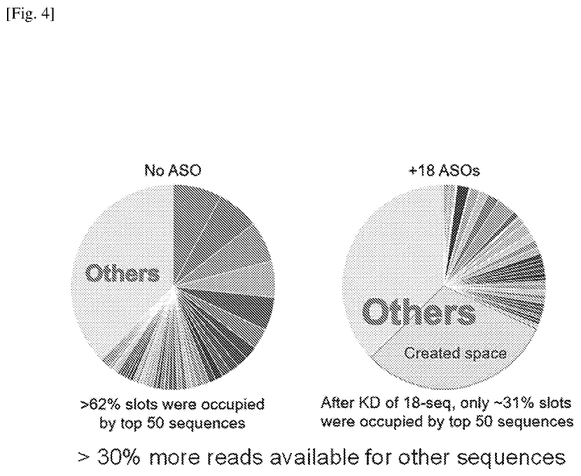

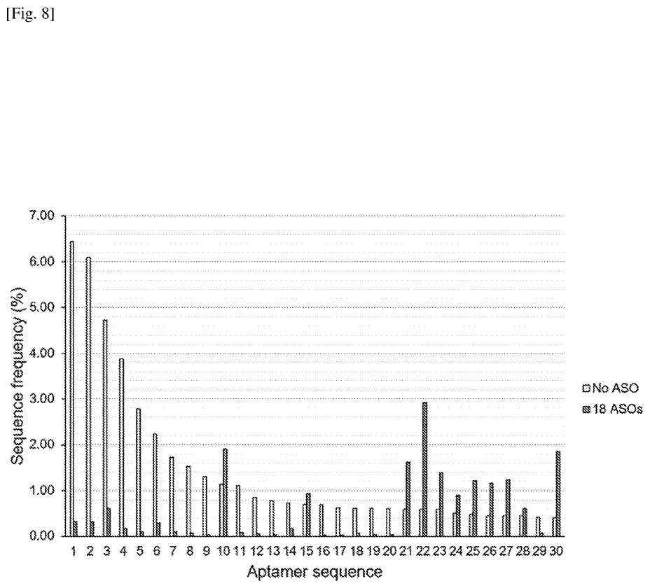

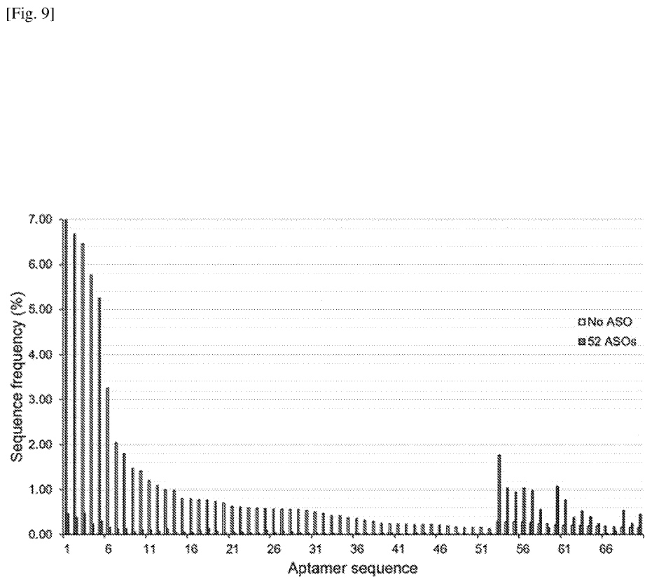

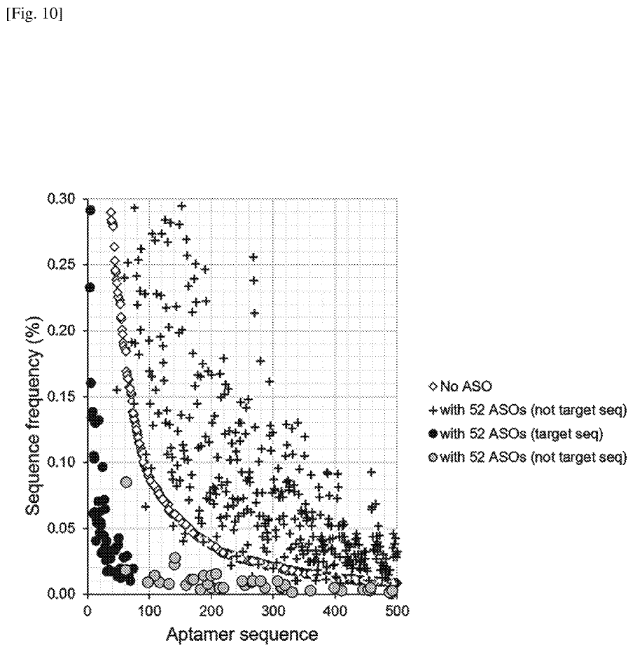

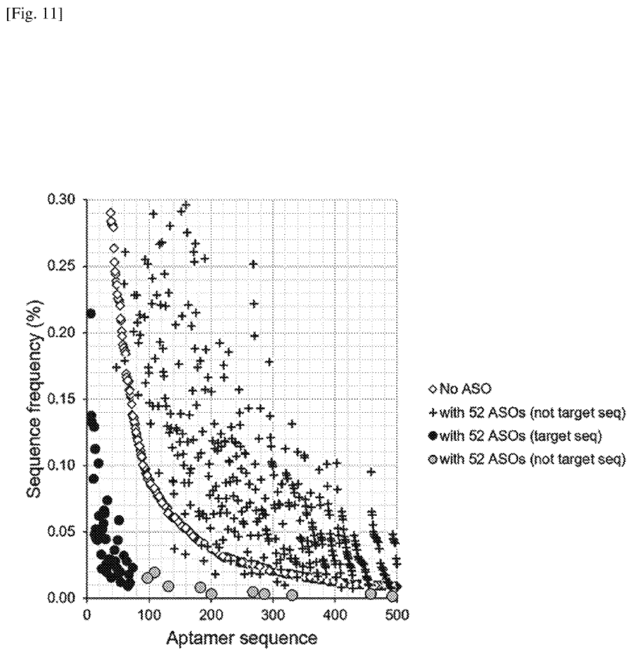

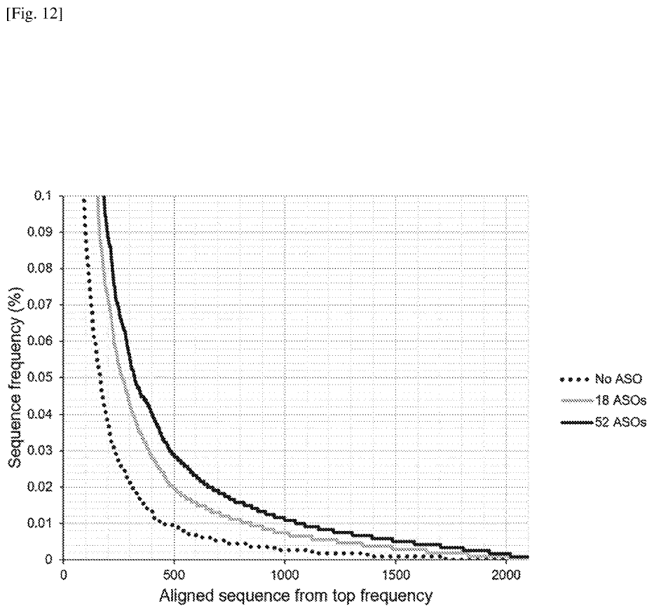

In a validation experiment, target aptamer sequences were selected from top frequency sequences observed in next generation sequencing analysis of the aptamer library. Two sequences were removed from the target list to investigate specificity of the invention. Antisense oligonucleotide library was systematically designed based on just sequence information obtained from next generation sequencing. The length was fixed at 30 nucleotides and the target sequence was set from position 7 of the aptamer domain. DNA oligonucleotides were chemically synthesized without any specific chemical modification. The results of simultaneous inactivation of 18 aptamers by 18 different ASOs is shown in FIG. 3 and FIG. 8. Aptamers #10 and #15 were not targeted by any ASO in this experiment to investigate ASO specificity. The results of simultaneous inactivation of 52 aptamers by 52 different ASOs is shown in FIG. 9. Changes in sequence frequency after the addition of ASOs were investigated. FIG. 10 shows change in sequence frequency in an aptamer library in which ASOs were added at the time of aptamer denaturation to hybridize ASOs with target aptamers. Sequence comparison analysis of the non-specific inactivation sequences by added ASOs revealed that non-specific inactivation occurred in relatively higher homology sequences with ASOs. FIG. 11 shows change in sequence frequency in an aptamer library in which ASOs were added after the formation of aptamer structures to reduce unexpected hybridization between aptamer and ASO due to high homology sequence. Analysis of number of available sequences after target depletion is shown in FIG. 12. It was revealed that around 30% more sequences were observed with enough sequence frequency for sequencing analysis (FIG. 4).

1. A method of sequencing low abundance aptamers from an aptamer library, the method comprising:
(i) amplifying a plurality of aptamers capable of binding to one or more target molecules in a sample by emulsion PCR to generate an aptamer library;
(ii) sequencing the aptamer library; and
(iii) contacting a plurality of antisense oligonucleotides (ASOs) targeting high abundance aptamers of the aptamer library with the aptamer library to form a mixture; wherein contacting the ASOs with the aptamer library results in inactivation of the high abundance aptamers of the aptamer library.

2. The method of claim 1, further comprising selecting the plurality of aptamers capable of binding to one or more target molecules in a sample in steps (a)-(c):
(a) contacting a plurality of candidate aptamers with a sample comprising one or more target molecules to form a composition comprising a plurality of aptamer-target molecule complexes;
(b) purifying the plurality of aptamer-target molecule complexes; and
(c) extracting the plurality of aptamers capable of binding to one or more target molecules from the aptamer-target molecule complexes.

3. The method of claim 2, further comprising repeating the steps (a)-(c) and (i)-(iii), and wherein the mixture obtained from step (iii) comprises the plurality of candidate aptamers when repeating step (a).

4. The method of claim 3, wherein the method is repeated at least 3 times.

5. The method of any one of claim 1, further comprising sequencing the aptamer library obtained from step (iii).

6. The method of claim 1, wherein the ASOs comprise modified nucleotides.

7. The method of claim 1, wherein sequencing low abundance aptamers from an aptamer library comprises next generation sequencing (NGS).

8. The method of claim 1, wherein the sample is a biological sample.

9. The method of claim 8, wherein the biological sample is serum, plasma, cerebral-spinal fluid (CSF), urine, amniotic fluid, bone marrow, bronchoalveolar lavage fluid, buccal swab, feces, gastrointestinal fluid, liposuction sample, saliva, milk, nasal swab, peritoneal fluid, semen, sputum, synovial fluid, tears, vaginal fluid, tissue biopsy, cell lysates, cultured cell, tissue sample, or in vivo endothelial cell.

10. The method of claim 8, wherein the biological sample comprises target molecules including nucleic acids, proteins, polypeptides, carbohydrates, lipids, or a combination thereof.

11. The method of claim 8, wherein the biological sample is not denatured.

12. The method of claim 1, wherein the high abundance aptamers in the aptamer library are aptamers having a sequence frequency level of higher than 0.05% in the sequencing reaction in step (ii).

13. The method of claim 1, wherein the high abundance aptamers in the aptamer library are aptamers having a sequence frequency level of higher than 0.1% in the sequencing reaction in step (ii).

14. The method of claim 1, wherein the high abundance aptamers in the aptamer library are aptamers having a sequence frequency level of higher than 0.15% in the sequencing reaction in step (ii).

15. The method of claim 1, wherein the high abundance aptamers in the aptamer library are aptamers having a sequence frequency level of higher than 0.2% in the sequencing reaction in step (ii).

16. The method of claim 1, wherein the high abundance aptamers in the aptamer library are aptamers having a sequence frequency level of higher than 0.5% in the sequencing reaction in step (ii).

17. The method of claim 2, wherein step (b) comprises subjecting the composition to electrophoresis in a first electrophoresis medium in a first direction to obtain a portion of the first electrophoresis medium that comprises the aptamer-target molecule complexes.

18. The method of claim 17, wherein step (b) further comprises subjecting the portion of the first electrophoresis medium to electrophoresis in a second electrophoresis medium in a second direction to obtain a portion of the second electrophoresis medium that comprises the aptamer-target molecule complexes.

19-35. (canceled)

36. The method of claim 1, further comprising denaturing and renaturing the aptamer library before contacting the ASOs with the aptamer library.

37. The method of claim 1, further comprising denaturing and renaturing the aptamer library after contacting the ASOs with the aptamer library.
